(19) United States
(12) Patent Application Publication
(10) Pub. No.: US 2023/0030373 A1
(43) Pub. Date: Feb. 2, 2023

(54) MIXSEQ: MIXTURE SEQUENCING USING COMPRESSED SENSING FOR IN-SITU AND IN-VITRO APPLICATIONS

(71) Applicants: Alexander G. VAUGHAN, Cold Spring Harbor, NY (US); Anthony M. ZADOR, Cold Spring Harbor, NY (US)

(72) Inventors: Alexander G. Vaughan, Cold Spring Harbor, NY (US); Anthony M. Zador, Cold Spring Harbor, NY (US)

(73) Assignee: Cold Spring Harbor Laboratory, Cold Spring Harbor, NY (US)

(21) Appl. No.: 17/788,603

(22) PCT Filed: Dec. 23, 2020
(86) PCT No.: PCT/US2020/066853

§ 371 Date: Jun. 23, 2022

Related U.S. Application Data
(60) Provisional application No. 62/953,174, filed on Dec. 23, 2019.

(51) Int. Cl. G16B 40/10; G16B 30/00

(52) U.S. Cl. CPC G16B 40/10; G16B 30/00

(57) ABSTRACT

Recently, advances in next-generation sequencing have arisen from the spatial isolation of each molecule into a small volume, enabling many single-molecule sequencing reactions to run in parallel. The fundamental limit to throughput with this technique is the need to isolate individual molecules on a spatial scale, so that sequencing signals are not mixed. Here we disrupt this limit, by observing that, in many cases, it is possible to accurately sequence complex mixtures of DNA and RNA species by exploiting the toolkit of modern compressed sensing and incorporating additional relational information about the relationship between many sequencing problems. This approach thus provides a dramatic increase in the density of DNA molecules in the sequencing reaction for both in-vitro and in-situ techniques.

This application claims benefit of U.S. Provisional Application No. 62/953,174, filed Dec. 23, 2019, the entire content of which is hereby incorporated by reference herein.

### GOVERNMENT SUPPORT

This invention was made with government support under grant number D16PC0008 awarded by the Intelligence Advanced Research Projects Activity (IARPA). The government has certain rights in the invention.

Throughout this application, various publications are referenced, including referenced in parenthesis. The disclosures of all publications mentioned in this application in their entireties are hereby incorporated by reference into this application in order to more fully describe the state of the art to which this invention pertains.

This invention will be better understood by reference to the detailed description which follows, but those skilled in the art will readily appreciate that the specific experiments detailed are only illustrative of the invention as defined in the claims which follow thereafter.

### BACKGROUND OF THE INVENTION

The advance of biology over the last half-century is inextricably tied to DNA sequencing. Although there are dozens of major technologies for sequencing, all rely on the serial identification of the four bases G/T/A/C from single molecules, and the spatial isolation of signals arising from each molecule being the main determinant of throughput.

Recently, advances in next-generation sequencing have arisen from the spatial isolation of each molecule into a small volume, enabling many single-molecule sequencing reactions to run in parallel. The fundamental limit to throughput with this technique is the need to isolate individual molecules on a spatial scale, so that sequencing signals are not mixed.

Disclosed here is a method to accurately sequence complex mixtures of DNA and RNA species, in such a way as to reveal the underlying sequences that make up the mixture. This approach provides for a dramatic increase in the density of DNA molecules in a sequencing reaction for both in-vitro and in-situ techniques.

### SUMMARY OF THE INVENTION

Aspects of the invention provided herein can be analogized to attempting to read a grocery list having overlapping text that is so densely written that two words are superimposed. For example, two overlapping words may appear as:

| | | | | |
|---|---|---|---|---|
| G + P | E + R | A + A | C + P | E + H |

Unraveling this mixture is difficult. However, a dictionary of 5-letter fruits is useful to determine the identity of each word. In the above example, the dictionary may contain the fruits: [APPLE, GRAPE, LEMON, MANGO, MELON, PEACH, PRUNE]. Applying logical deduction or a combinatorial search reveals that the mixed signal provided above can be resolved only one way:
PEACH+GRAPE.

This problem can also be framed as a linear algebra problem. (See FIG. 1). Perhaps surprisingly, this problem, and the algebra underlying its solution, has a direct analogy to the problem of high-throughput DNA sequencing. Currently, the throughput of DNA sequencing is limited by the need to physically isolate each sample, to ensure that the resulting sequence information is not mixed. This physical isolation determines the physical density, and thus the overall throughput, of multiplexed sequencing. Indeed, because mixed sequencing information has previously been viewed as unusable data, sequencing platforms have developed patterned flow cell technology and exclusion amplification chemistry in order to increase monoclonal clustering and prevent generation of mixed sequencing signals.

This invention provided herein demonstrates that it is possible to break this barrier, and to generate accurate sequencing information while operating in a heavily mixed regime. To resolve these mixtures, a toolset of compressed sensing is used, which seeks to identify the simplest or ‘sparsest’ solution to mixed signals, and efficiently ‘demixes’ the ambiguous original sequence information. This approach, which we term Mixture Sequencing (MIXSEQ), enables a massive increase in sequencing throughput for a variety of traditional sequencing platforms, as well as a new class of experiments for in-situ sequencing.

In comparison to the grocery list problem above, the problem of high-density DNA sequencing differs in form in a few important ways. First, there are only four letters (A,T,G,C) instead of 26. Second, a raw sequencing signal is not as categorical as letters from an alphabet, but instead varies in amplitude, and is contaminated by noise. Third, it is not always obvious how many signals are mixed together, and this must be determined in the process of demixing the signal. In general, however, these differences are addressed within the compressed sensing framework.

Importantly, the approach described here reframes the sequencing operation from a search for de novo sequences, into a regression problem in which sequences are matched to an existing dictionary.

Depending on the biological problem, this dictionary may represent a transcriptome, genome, a set of random DNA barcodes, a set of RNA or DNA aptamers, or any other set of oligonucleotides with biological relevance.

The form of this dictionary is important. Suppose one attempts to decode our ambiguous grocery signal described above—G+P, E+R, A+A, C+P, E+H—using the full English dictionary instead of a simple dictionary of fruits. Suddenly, two equivalent solutions may be found: two fruits (PEACH+GRAPE), or two generic nouns (PEACE+GRAPH). This kind of ambiguity also affects the DNA sequencing problem and arises directly as a function of dictionary size. In general, larger dictionaries make the demixing problem more difficult. However, as shown herein, the relevant dictionaries for a wide range of biological problems—including transcriptome sequencing, genome sequencing for CNV analysis, and single-cell barcoding—are readily available or can be readily applied. Moreover, multiple dictionaries can be applied individually to the same data set and the results can be compared for differences which in turn can be used to decide which is the most probably correct result.

To properly frame the problem solved by the MIXSEQ approach, the general process of DNA sequencing in its current form is outlined. Typical approaches to DNA sequencing typically have three steps: (1) isolation of a single molecular species, (2) selective amplification and (3) performance of the actual sequencing reaction through repeated measurement. The exact sequencing method varies, but common methods such as Sanger or sequencing by synthesis e.g., Illumina, typically return a 4-channel measurement corresponding to each possible base at a given nucleotide position.

As an example, consider modern next-generation sequencing on the Illumina HiSeq platform. Individual molecules from the DNA sample are first isolated onto a glass flow cell, and then amplified to form many small colonies. This colony is subjected to “sequencing by synthesis,” in which the sequence is read via successive incorporation of fluorescent nucleotides. Using this platform, sequencing information is read out through 4-channel fluorescent microscopy by identifying the fluorescent signal associated with each colony.

The massive throughput enabled by this method is enabled by the high density of the DNA colonies, and the ability to multiplex millions of colonies on a single coverslip. Nonetheless, this density is limited by the need to avoid generating colonies at too high a density, because over-lapping colonies generate ambiguously mixed sequence information. This limitation arises because of the impossibility of performing traditional base-calling on mixed sequence information.

Modern methods for in-situ sequencing suffer from a similar problem. For instance, Fluorescent In-Situ Sequencing (FISSEQ) is a method for transcriptome sequencing that relies on transforming each RNA molecule into a small amplified RNA colony (“rolony”) in the physical context of the original cell. These rolonies can then be sequenced using fluorescent chemistry. The efficacy of this method is ultimately limited by rolony density, as overlapping rolonies provide an ambiguously mixed signal.

To move beyond this barrier, “mixture sequencing” (MIXSEQ) replaces the traditional base-calling step with an algorithmic demixing of overlapping signals. This approach operates directly on the superimposed fluorescent signal arising from multiple DNA sequences.

In order to resolve these signals, MIXSEQ relies on previous knowledge of a dictionary of known sequences, such as a previously sequenced genome or transcriptome. In many cases, the dictionary can also be selected based on the data itself.

By enabling the identification of sequence information from mixed samples, MIXSEQ allows for a new form of multiplexed sequencing that enhances the throughput of both in-vitro and in-situ sequencing and allows for new biological experiments in in-situ sequencing methods.

To further demonstrate the utility of applying MIXSEQ to in-situ sequencing methods, FISSEQ is first described here in more detail. Using the traditional FISSEQ technique, endogenous mRNAs are subjected to a three-step process. First, endogenous RNAs are subjected to reverse transcription, forming a short complementary DNA (cDNA) containing the “target sequence”. Second, the target sequence is selected and incorporated onto an exogenous nucleic acid backbone either via a gap-filling ligation using a padlock probe or via circLigase. Third, the circularized product including the target sequence is amplified via rolling circle amplification using Phi29 polymerase, which generates a rolling circle colony or “rolony”. Fourth, the target sequence is selectively read out by application of a flanking sequencing primer and well-known sequencing methods, such as the chemical processes involved in sequencing by ligation (SBL) or Illumina sequencing methods. This gives rise to a “sequencing signal”. Fifth, the sequencing signal is subjected to standard base-calling methods, which seek at each position to find the most likely nucleotide in the target sequence. For standard sequencing using four-color fluorescent methods, this base-calling happens by identifying the color channel with maximum intensity.

Theoretically, every RNA molecule in the sample is assumed to give rise to a maximum of one rolony, and one associated nucleotide sequence known as the target sequence. While some target sequences may be present in multiple rolonies, each rolony only contains one target sequence. Therefore, the base-calling algorithm may be run on each 2-dimensional pixel or 3-dimensional voxel individually or may be run on a collection of pixels such as an entire identified rolony. This base-calling algorithm operates by identifying and interpreting the fluorescent intensities arising from the sequencing operation (the sequencing signal) and assigning a single nucleotide base for each position in the molecule according to the relative fluorescent intensity in each channel.

A major practical limitation of FISSEQ is that in order to run standard base-calling algorithms, the set of rolonies in the sample must not overlap physically. Indeed, currently significant effort is taken to avoid rolony overlap. Rolony overlap can be avoided through several processes including (a) sequencing relatively few target sequences at a time (Ke et al., 2013), (b) physically expanding the tissue using “expansion microscopy,” (Chen et al., 2016), (c) reducing the physical size of rolonies, at the expense of a dimmer signal and less-reliable base-calling, (d) sequencing only a subset of rolonies in a given sample by careful selection of sequencing primers, or (e) improvements in microscopy, at the expense of imaging time. Each of these methods has costs in terms of the number of sequenced molecules, signal-to-noise ratio, imaging time, etc.

The need to prevent rolony overlap provides a fundamental limit to the usefulness of FISSEQ, because the size of the cell is quite limited relative to the size of a single rolony. For example, a spherical cell of 5 μm radius can only contain ˜1000 spherical rolonies of radius 0.5 μm. This number is insufficient to support many uses of FISSEQ, such as robust quantification of mRNA copy number for more than a small number of genes.

The inventive method described here provides an alternative method for addressing the problem of rolony overlap and increases the overall throughput of the sequencing reaction.

First, we alter the method of FISSEQ to intentionally generate rolonies with high levels of overlap. This overlap may be quantified by the average distance between rolonies, such that at least 5%, 10%, 25%, 50%, 90%, or 100% of rolonies are within 0.5 μm 1 μm, 2 μm of its nearest neighboring rolony. In another method of quantifying overlap without careful distance measurements, rolonies may be considered to overlap if, for at least 5%, 10%, 25%, 50%, 90%, or 100% of rolonies, at least 5%, 10%, 25%, 50% 90%, or 100% of the pixels imaged for that rolony overlap with pixels from another rolony.

In the process of sequencing, the overlap of two or more rolonies gives rise to a “mixed sequencing signal,” which may contain information from two or more target sequences. For a given pixel or collection of pixels, the mixed sequencing signal may represent the summation of sequencing signals for two or more rolonies, either in equal proportion, or in unequal proportions. Using traditional base-calling, it is not possible to “demix” this signal to identify the original target sequences.

However, using MIXSEQ it is possible to “demix” the mixed sequencing signal to identify the set of target sequences that are present in overlapping rolonies. This method can be applied either to individual pixels in the image or it can be applied to collections of pixels. In both cases, we will refer to the input data as the “mixed sequencing signal.”

As described in this application, in order to identify the target sequences present in the set of overlapping rolonies that form the mixed sequencing signal, the signal is compared to a known dictionary, i.e. a database of known nucleic acid sequences that are potentially expressed or contained within the sample. In this application, such a dictionary is referred to as the “sequence dictionary,” and it can be drawn from, for example, known sequences from the transcriptome or genome of any species. The sequence dictionary may also contain a set of apparently random sequences of known length, e.g. “barcodes,” that may arise from exogenous sources such as virus infection or direct transfection and are found within a tissue as RNA or DNA molecules.

For the demixing process, the goal is to identify a combination of target sequences that may adequately reconstruct the mixed sequencing signal. This combination is referred to as the “demixed solution” to this problem and defines a set of weights or probabilities for each sequence in the sequence dictionaries, with these values corresponding to an estimate of the proportional contribution (or probability of contribution) to the mixed sequencing signal. This demixed solution can be found using a variety of algorithms, including those of regression, constrained regression, LASSO, combinatorial theory, compressed sensing, compressive sensing, convex optimization, approximate message passing, belief propagation, logistic regression, deep learning, and others.

One useful approach is to seek a demixed solution that is “simple” in some way. In the context of the demixed solution, “simplest” may suggest that the smallest number of target sequences are used, or that the relative weights of several target sequences are relatively low, measured as the average weight, maximum weight, L1 weight, L2 weight, entropy of weights, etc. This approach is commonly used in other fields that seek to “demix” other signals such as image processing, radar, etc.

In the context of the demixed solution, a reconstruction may be understood as “adequate” if it is sufficient to explain most of the variability, or amplitude of the mixed sequencing signal, with error that is less than 90%, 75%, 50%, 25%, 10%, 5% or less.

As the mixed sequencing signal may consist of signals from many pixels, the weights applied to each pixel may be different. The measurement of a “simple” solution may also be combined across pixels. For example, the solution to a many-pixel problem may be found by a “Group LASSO” or “multiple Gaussian” solver. Additional relational information about the many-pixel problem may arise, for example, if pixels are arranged spatially such that nearby pixels are likely to carry similar signals. Additional relational information about the many-pixel problem may also arise if groups of target sequences within the sequence dictionary are likely to show correlations in their presence or absence across pixels. Lastly, additional relational information about the many-pixel problem may be used when the goal is to identify deviations from the dictionary—for example, using the solutions of many such sequencing problems to identify deletions or single nucleotide polymorphisms (SNPs) in the target sequences.

### DETAILED DESCRIPTION OF THE INVENTION

The outline of the MIXSEQ approach has three parts: (1) a mathematical framework for sequence demixing using compressed sensing; (2) delineation of the limits of MIXSEQ and heuristics for identifying solvable problems; and (3) practical applications of this technique for sequencing genomes, transcriptomes on Illumina or FISSEQ platforms.

### (1) A Mathematical Framework for Sequence Demixing

The MIXSEQ approach essentially replaces the traditional base-calling step of DNA sequencing. Traditionally, base-calling operates on a signal from one DNA species, consisting of an analogue value for each possible nucleotide i.e., G/T/A/C, at each position. The MIXSEQ approach replaces this step, and instead enables the processing of superimposed signals from many DNA species. To outline the MIXSEQ approach, the problem of base-calling must first be framed in terms of linear algebra (FIG. 2, FIG. 3, FIG. 4).

For a general, non-sequencing related problem, a set of n measurements can be made and represented as a vector s. It is known that s is made up of several superimposed signals with differing features, drawn from a dictionary A consisting of p dictionary elements. Thus, s is a weighted sum of the elements, or columns, of A. We can write the problem as a simple regression problem:

s=Ax Equation 1:

Here x is the unknown set of weights or loadings that denote which dictionary elements i.e., columns of A, have been mixed into the measurement s. Also, note that the problem here is simplified such that each measurement is a one-dimensional scalar, rather than a 4- or 26- dimensional vector.

Using traditional linear algebra, we can solve for x as long as the number of measurements (n) is at least as large as the dictionary size (p), assuming A has full rank. However, this regime of n>=p is less useful for our purposes, as it requires a very small dictionary. The more likely regime is n<<p. Here, however, the problem is underdetermined—that is, it has infinitely many possible solutions.

The fundamental result of compressed sensing is that underdetermined problems can indeed be solved, as long as long as the solution is “sparse”—that is, made up of relatively few underlying components.

For the grocery list problem described previously, this is analogous to saying that a solution that combines two 5-letter words e.g., PEACH/GRAPE is preferred over one that combines several smaller words or letters e.g., the 5-component mixture [PEA--+--APE+G----+-R---+---C-+----H.]

Several methods are available to identify the “sparsest” solution.

The most obvious is to search over all combinations of dictionary elements in A. The sparsest solution might then be found by minimizing the number of non-zero elements in x—this is known as the L0 norm of x, written |x|_o. While effective, this is computationally impractical for all but the most trivial problems.

It has been shown that the sparsest solution can also be identified by minimizing the L1 norm of x, corresponding to the summed magnitude of elements of x. This approach identifies the same solution as the L0 norm, but is computationally tractable and efficient. A variety of algorithms are available for this approach and are used, for example, in radar, JPEG compression, and MRI (Blanchard, 2013). Approaching the large-dictionary problem with L1-norm minimization or related convex problems has proved to be a powerful and general method for resolving ambiguous mixtures.

To adapt this approach to the problem of DNA sequencing, we must rewrite the measurement s to match the form of the signal coming from the sequencing machine. We start with a single molecule of DNA of length c. Under four-color fluorescent sequencing, the sequence of this molecule would be read as a n=4c sequence matrix S. Each row of S corresponds to a single nucleotide position, and each column corresponding to a color channel associated with nucleotide A/T/G/C. (FIG. 3). Using traditional base-calling, the appropriate base is identified from the channel with maximum intensity.

However, for a mixture of molecules in a sequencing reaction the fluorescence output at each nucleotide position will consist of multiple colors and the sum S* of two or more sequencing matrices i.e., S*=S1+ . . . +Sn, will be observed. (FIG. 4). To solve this problem, a regression problem is outlined in which the signal S* is defined as an unknown mixture x of elements from a dictionary A of known DNA sequences relevant to the biological problem. We then reshape S* into a vector s* of length n=4c and reframe the problem as a regression:

s*=Ax(1) Equation 2:

Here A is a dictionary of p known sequences relevant to the biological problem. As before, this problem is trivially solvable if n>=p. Modern sequencing technology restricts the size of n to approximately 1E2-1E3. What then, is the size of p?

In the worst-case scenario, the dictionary would contain every possible n-mer, and we would seek the sparsest solution using combinatorial search. If we sequence only n nucleotides, there are a total of p=4n possible sequences and ˜4kn combinations of k sequences. Analyzing this problem combinatorically is clearly infeasible: for a measurement of length n=20, testing one pairwise combination per nanosecond would require nearly a billion years. Fortunately, two factors coincide to render this problem more tractable.

The first is that, in typical biological applications, dictionary size is much smaller than 4n because only a subset of possible n-mers are relevant to the problem. For example, of all 4°˜1E12 nucleotide sequences of length 20, less than 0.4% (p˜1E10) are actually used in the human genome. (Liu et al., 2008). In the case of transcriptome sequencing, appropriate dictionaries can be built on the order of the number of genes (p˜1E5-1E6). Or, for truly random sequences such as those used for tissue barcoding, p is a directly tunable parameter (p˜1E4-1E10 for neural barcoding). Thus, the size of the working dictionary is much more manageable than at first glance.

Our second result is mathematical, arising from the field of compressed sensing. An important result is that the “sparsest” solution as measured by the L0 norm is, in many cases, also the sparsest when measured by the L1 norm. This problem can then be solved, as before, by seeking solutions that minimize the L1 norm of X.
That is, we solve: argmin(x) |x|_1 s.t. s=Ax
Or, accounting for noise: argmin(x) |x|_1 s.t.|Ax−s|<eps

Identifying the solution with the smallest L1 norm is significantly easier than the L0 norm because this problem is convex—any locally optimal solution is guaranteed to be the global optimum as well. In most cases and as assessed in detail below, the L1 solution is also the same as the L0 solution and can be found using a variety of algorithms that are efficient, robust, and resistant to noise.

To provide a flavor of these algorithms, one algorithm known alternately as Matching Pursuit or stepwise regression is briefly outlined below. It proceeds as follows:
1) Identify the single dictionary element in A that has the highest correlation with s. Define this as the active set {A*};
2) Calculate the minimal residual using only the elements of the active set, i.e., r=s−{A*}x, by solving for x;
3) Identify the element of A that has the highest correlation to r, and add it to the active set {A*};
4) Repeat 3-4 until the magnitude of the residual, |r|, is sufficiently close to 0.

In practice this procedure will identify one DNA sequence from A for each iteration, repeating until the mixed signal is adequately explained.

To demonstrate the efficacy of this technique, a dictionary of 10,000 random barcodes was generated and used to attempt to resolve a mixed sequencing signal of 25 nucleotides generated from 10 randomly chosen barcodes. (See FIG. 6). Using the algorithm above, this procedure identifies the correct components with nearly 100% efficiency, as long as the number of measurements (n) and noise level (SNR) are above a given threshold. The bimodal success rate is common for this approach.

In the next section, the conditions under which this approach is likely to be successful is outlined and it is shown that many biological problems lie within the feasible range.

(2) Determining when the MIXSEQ Problem is Solvable in the Absence of Noise

The field of compressed sensing has devoted considerable energy to understanding the circumstances under which underdetermined problems (such as Equation 1) can be efficiently solved and when they find the right answer. A major result is the observation that the solvability of a compressed sensing problem shows a phase transition—shifting between “almost always” solvable and “almost always” insolvable—that depends on the size of the problem. This is defined by three parameters: number of measurements n, the size of the dictionary p, and the number of mixed components k in the final solution. (Donoho and Elad, 2003). In general, problems are solvable for relatively large n, relatively small p, and relatively small k. This result is remarkably general and provides a roadmap for ensuring that any given problem is solvable.

To begin an analysis of this problem, we return to our one-dimensional problem (Equation 1) in which the dictionary A is derived from random Gaussian measurements, the k non-zero loadings in x are all equal in magnitude, and there is no noise. Problems of this form have the most permissive bounds for solvability.

To understand the behavior of this problem, two parameters are defined. First, the sparsity fraction (delta=k/n), which is the number of non-zero coefficients in x per measurement (n). The under-sampling rho is also defined, which is the number of measurements (n) divided by the dictionary size (p). When rho=1, the problem is no longer underdetermined and is always solvable.

As an example, we then generated a random dictionary of size p=1000 and simulated the results of 10,000 simulations. The results show a remarkable phase transition in solvability. Following Donoho, we report both the L2 error in the coefficients (that is, ∥X−X*∥) as well as the probability that this L2 error exceeds a small tolerance (eps=1E-5). (See FIG. 7; Donoho and Elad, 2003). It is clear that the problem is successful when the delta is small, and when rho is large. This data is also plotted in log scale (FIG. 7, right) with markings to show the actual values for n (purple, top axis) and k (gray, right axis). It is clear that, for a given dictionary size, an infeasible problem can be rendered feasible by either (a) decreasing k, or (b) increasing n.

Importantly, however, the success or failure of the algorithm here is not easily determined without knowledge of the ground truth {x}. Methods for assessing these errors are discussed below.

The location of this phase transition for different types of dictionaries is examined next. The problem is optimal when the dictionary has maximum entropy, as for the Gaussian distribution. However, similar dictionaries with less entropy, including the Bernoulli dictionaries, consisting of random numbers drawn from [0,1], show similar results. (FIG. 8, center). Indeed, dictionaries consisting of random DNA n-mers (i.e. barcodes), which are somewhat different because they are 4-dimensional, show a qualitatively similar phase transition to the Gaussian case.

The dictionary of DNA barcodes is indexed somewhat differently than others, because the 4-dimensional measurement for each nucleotide is concatenated into a single vector. These bounds are somewhat clearer when indexed by the number of bases sequenced. (See FIG. 8). In Figure. 8, the bounds for a dictionary of 10,000 DNA barcodes for reasonable sequencing lengths appropriate to FISSEQ (˜20 nucleotides) and Illumina sequencing (˜150 nucleotides) are plotted. For this problem, we see that between 8- and 40- mixed sequences can be reliably resolved.

One important feature of the problem is not incorporated into this representation—dictionary size (see FIG. 9). The grocery list example showed that larger dictionaries give rise to greater ambiguity, and indeed that holds here. From a practical standpoint, increasing the dictionary size by 10-fold results in a 2-fold decrease in the maximum resolvable k. If this slope is parameterized as c=log(k/m)/log(m/n), the value for this particular algorithm and problem size is c=0.2. This exact number will vary somewhat depending on the algorithm used. Also, as discussed below, this threshold can be increased somewhat by using side-channel information, such as signals from nearby pixels.

(3) Resistance to Noise

The MIXSEQ approach described herein also shows a remarkable resistance to noise (see FIG. 10). Interestingly, this property is shared across any method that performs a “best-match” projection of the data onto a dictionary. As an example, we generated a series of synthetic problems in which we sequenced between 1 and 500 bases, with each synthetic sample consisting of an equal mixture of between 1 and 128 molecules. We then generated noise sufficient to reduce the Q-score [defined as −10 log₁₀ (p) where p is the probability of an error at each nucleotide position] of between 20 and 50. For the Illumina platform, Q-scores of between 30 and 40 are common. Using our approach, we observed that we could reliably “demix” approximately 4 sequences when we sequenced 100 base pairs, increasing up to approximately 8 sequences when we sequenced up to 500 base pairs. These simulations were performed using a dictionary of 10,000 random barcodes, with demixing accomplished on a single-pixel basis using non-negative Orthogonal Matching Pursuit.

(4) Estimation of the False Positive Rate

For any practical application of this technique, it will be important to estimate the false detection rate. In this context the false detection rate is defined as the probability that, for a problem of a given size solved by a given algorithm, the correct dictionary sequence will be chosen. This probability can be approximated by adding a set of “bait” sequences to the sequence dictionary, which are known not to correspond to any biological sequence. Assuming that the dictionary is random, the likelihood that the demixing procedure will choose any given “bait” sequence is equal to the probability of choosing false-positive within the original sequence dictionary. As the inclusion of such “bait” sequences always decreases the overall probability of successful recovery, this FDR can be considered to be a conservative estimate.

(5) Exploiting Multi-Pixel Correlations

The method used in Equation 2, s=Ax, is actually a simplification of the sequencing signal. Typically, for either Illumina or FISSEQ sequencing, in which the signal is a multi-channel fluorescent signal observed through fairly traditional microscopy, there are strong correlations between neighboring pixels. This correlation can be exploited by altering the approach used in Equation 2 to enable a multi-pixel decoding, encouraging solutions that use the same dictionary elements across pixels. Here it is assumed that the measurement vector s is repeated, forming a measurement matrix S—each column corresponding to an individual pixel. Similarly, the weight vector x is expanded to a weight matrix X with each column corresponding to the weights of each dictionary sequence for a given pixel. It is assumed that the same set of non-zero weights in X are shared across pixels, although the magnitude of each may vary for each pixel. Typically, it is also assumed that these weights are independent and uncorrelated across pixels, however, this assumption is frequently violated in real data to little detriment.

Thus, the problem can be reframed such that the reconstruction error of S=AX is attempted to be minimized while identifying the sparsest set of weights X. We encourage the use of the same barcode across pixels calculating the L2 norm of each barcode (across pixels), and subsequently calculating the L1 norm of total barcode loadings. This reduces the penalty for using the same barcode across multiple pixels, encouraging the use of similar barcodes.

min(∥AX-S∥) s.t|X|_{1,2}<epsilon Equation 3:

Similarly, it is also possible to encourage solutions in which there is explicit spatial smoothness i.e., neighboring pixels are similar. We define f(X) as a measure of the spatial variation in X, and then solve the equation:

min(|X|) s.t.∥AX−S∥<epsilon AND f(X)<epsilon Equation 4:

Although both approaches are algorithmically complex, either can be solved using the flexible toolkit of convex optimization.

These approaches have two uses. The spatially smoothed approach is useful when the actual physical measurement e.g., the signal arising from fluorescent microscopy, is spatially smooth; it exploits this smoothness to more reliably identify the correct sparse solution to the mixing problem. For the non-spatial approach, which simply encourages the consistent use of a common set of dictionary sequences, the assumption is that there is an intrinsic structure to the sequences themselves. This might be appropriate, for example, when identifying species communities, from a collection of multiple mixed sequence signals that independently or differentially subsample the underlying population. (Amir and Zuk, 2010). We note that this work takes a similar approach to that described here but is restricted to a single measurement of one mixed sequencing signal.

(6) Identify SNPs by Reliable Deviation from the Dictionary

In many cases, the biological question of interest involves deviations from an expected sequence, particularly including single nucleotide polymorphisms (SNPs). Intuitively, the presence of a SNP seems likely to derail the demixing process, as there is no correct dictionary match. In practice, however, the SNP variation will not disrupt identification of the correct template from the sequence dictionary.

Indeed, this approach can even be exploited to identify the presence of the SNP, without actually performing base-calling at any step (FIG. 11). Consider the case in which there are a large set of q subproblems that have been solved using the demixing procedure, with each measurement corresponding to a column of the measurement matrix S. To identify potential SNPs in a dictionary element c, one approach is to identify all of the problems in q that have a non-zero coefficient for c and to examine the residuals for outliers that correspond to the unknown SNP.

More generally, we can define our problem as Eq 3: S=AX. With SNPs, the correct problem uses a slightly different, but unknown, dictionary ASNP; thus Eq 4: S=A_{SNP}X_{SNP}. The set of SNPs that distinguish Â from A is unknown. However, this problem can be rewritten as S=(A+Δ)X_{SNP}, where Δ=A_{SNP}−A. Assuming that the effect of SNPs on the resulting solution X and measurement vector S is small, then X≅X_{SNP}, and the associate property allows for S=AX_{SNP}+ΔX_{SNP}. This can be solved piecewise by (1) solving S=AX; (2) calculating the residual R=S−AX; and (3) solving R=ΔX_{SNP} for unknown Δ.

(7) Learning the Dictionary

Identifying SNPs is a specific case of the more general problem of learning the full dictionary A. Given the ability to exploit correlations in the signal between neighboring pixels, it is often possible to learn the dictionary directly. In this sense, the set of pixels showing mixed fluorescence can be thought of as delineating a subspace that is spanned by a few unknown sequences. These can be learned using a variety of subspace estimation algorithms that are similar to principal components analysis (PCA). For example, both Non-Negative Matrix Factorization, Independent Components Analysis, and an appropriately formed and trained neural network can effectively identify the correct dictionary sequences.

For non-negative matrix factorization, we attempt to learn both the dictionary matrix A and weight matrix X. We do this by solving a related problem S=WH, where W is a dictionary representation and H is the weight matrix. One important bound placed on this problem is that all entries in W and H are constrained to be non-negative. This problem and variants can be reliably solved to identify the set of sparse components W that can be combined via the weight matrix H to reconstruct the signal S.

In the problem shown in FIG. 12, we attempt to identify 10 short barcodes (10-mers) from an image in which the 10 barcodes are distributed between 1000 colonies. Each colony is a small point, but standard imaging conditions have been simulated via a Gaussian blur. Two algorithms (Non-negative Matrix Factorization and Independent Components Analysis) successfully recover the sequences with slightly different degrees of success. Examples of this approach on biological samples are shown in FIG. 13 and FIG. 14.

Importantly, a variety of such algorithms exist, and can exploit (a) explicit assumptions about spatial smoothness, (b) cross-validation strategies for identifying sequences, and (c) terms that encourage further sparsity in both W and H. One important aspect of this approach is that, in general, the matrix W is not explicitly constrained to have the form of a traditional DNA sequence. While a simple solution is to run base-calling on each sequence in W to identify the nearest valid DNA sequence, it is also possible to constrain the algorithm to search only in the space of valid sequences.

One important aspect of dictionary learning techniques such as NMF and ICA, is the need to estimate the total number of sequences to learn. For this case, two methods are available—the L-curve (originally introduced in the context of ridge regression, (See FIG. 14) and cross-validation. Both are effective for this problem.

(8) Deviations from the Assumption of a Random Dictionary

The reformulation of the sequencing problem via compressed sensing carries an important assumption about the form of the sequence dictionary. This assumption usually states that the dictionary is drawn from a Uniform Spherical Ensemble—that is, that dictionary elements can be understood as points on a unit sphere, distributed uniformly on that sphere. (See, e.g., Donoho et al., 2006).

In the original motivation for this work—DNA barcoding—the barcode dictionary consists of barcodes that are approximately random at each base. This is sufficient to provide for a Uniform Spherical Ensemble.

Typical synthesis procedures for barcode generation do not ensure equal distribution of each base at each position. This decreases the overall entropy of the barcode library and reduces the information gained in each round of sequencing. However, the magnitude of this effect is relatively small. For a perfectly random barcode library, we gain 2 bits per nucleotide; if one base is left out entirely, this drops to 1.6 bits. If each nucleotide in the mixture is drawn uniformly from a range of 0×-2× the target value, we receive 1.75 bits of information. Indeed, simulations suggest that for sequence dictionaries derived from existing genomes or transcriptomes, the dictionary is likely to deviate even further from random. For every such dictionary, a confusion matrix can be generated to identify the probability that two sequences will be swapped during the demixing procedure.

(9) Recovery of Copy Number Variation

In many cases, it is desirable to treat the recovered sequences as arising from a single linear chromosome and to identify duplications and deletions in this chromosome. This problem is similar to the circular binary segmentation problem, (Olshen et al., 2004), but, in this case, must be recovered from data in which all sequences are intermixed. We have recently demonstrated that this is possible through an additional constraint on the MIXSEQ approach, as discussed in detail below.

Firstly, copy number variation (CNV) is an important contributor to heritable and acquired genetic disorders such as cancer. However, analysis of copy number variation is expensive at the level of sequencing because each genome position must be sampled multiple times (30x+) in order to reliably recover its overall prevalence. As described herein, mixture sequencing (MIXSEQ) is an approach in which multiple DNA molecules are sequenced simultaneously, giving rise to superimposed signals that must be disambiguated using the toolbox of compressive sensing. This method lowers the overall cost of resequencing and is particularly well suited to the problem of copy number variation. Here, we outline the application of MIXSEQ to investigation of copy number variation. In particular, we attempt to derive a method in which a smoothed, or piecewise linear, estimate of CNV can be derived directly from the compressive sensing problem.

A model is derived from an extension of the degenerate oligonucleotide primed polymerase chain reaction (DOP-PCR) approach to linear whole-genome amplification and standard Illumina sequencing. In this technique, a degenerate primer is used to linearly amplify a small fraction of the genome for sequencing and can be used through various techniques for highly reliably CNV calling. (Wang et al., 2016). The small subset of the genome that is amplified using this technique is relatively small, e.g. 20,000 sequences, and can serve as a reasonable dictionary in a compressed sensing framework. It is also assumed that the sequencing operation is similar to standard 4-color Illumina sequencing, with many molecules sequenced across many thousands of pixels/clusters.

Under the assumption that the genome shows copy number variation, some fraction of subsequences along each chromosome will deviate from diploid copy number. Typical CNVs include duplications and deletions that lead to triploid/monoploid states, although more dramatic changes are possible.

The set of m sequences amplified by DOP-PCR is defined as the columns of a matrix A(_{n;m})). Sequences of length n in A (indexed as A.,ₘ for column m) consist of length-4n sequences with bases chosen randomly at an A+T:G+C ratio of 0.5. A single sequence s=‘GTAC’ then has the sequence vector representations=[A1 . . . A4, T1 . . . T4, G1 . . . G4, C1 . . . C4]T=[1 0 0 0, 0 1 0 0, 0 0 1 0, 0 0 1]T (here, commas are for convenience only). Although rows if n are not strictly independent in this case, this is ignored here. In addition, it is assumed that sequences in A are ordered and evenly spaced along a single linear chromosome.

The loadings of each sequence in A across p pixels are denoted as X(m,p). To account for copy number variation, a reference vector c is defined as a bimodal stairstep alternating between diploid, triploid, and monoploid states (See FIG. 15A). For each pixel p we assume that the set of non-zero coefficients for that pixel X.,p is sampled as Poisson(k) and that the probability of a non-zero loading for sequence m is p(xₘ>0)∝cm (with unit loadings).

We seek to estimate “Copy Number Variation” as c but, due to Poisson sampling, can only recover the noisy estimate we might call “Sequence Number Variation” s, where ŝₘ≡∥Xₘ,.>0∥₀. This is the quantity recoverable using traditional sequencing methods, in which there is one sequence per pixel (i.e., k=1) and traditional base-calling and sequence mapping may be used to populate X. After identifying the s by counting the occurrence of each sequence that maps to the genome, c is estimated as a piece-wise constant approximation to s by circular binary segmentation. (Olshen et al., 2004). The goal is to identify piecewise-constant subregions of the chromosome that share a common copy number, with well-defined edges.

Our problem deviates from the traditional approach in two ways. First, it is assumed that k>1, and that the estimate \hat{X} must be derived by a sparse inversion, taking for granted that this is feasible on both a physical and algorithmic basis. Second, it is noted that correct recovery of \hat{X} allows an estimate of Sequence Number Variation s that could be used to approximate Copy Number Variation c via circular binary segmentation. However, we assume that it is both possible and desirable to estimate ĉ directly by smoothing the estimate contained in \hat{X} using a variant of Fused LASSO. (Tibshirani et al., 2005).

We begin by framing the problem as a multi-task LASSO, (Obozinski et al., 2006), or the related multiple measurement vector Basis Pursuit problem (MMV-BP):

$${min}_{x} \frac{1}{2} {‖ Y - AX ‖}^{2} + lamda {‖ \hat{X} ‖}_{2 , 1} {min}_{x} {‖ \hat{X} ‖}_{1 , 1} s . t . {‖ Y - AX ‖}_{2 , 2} < sigma$$

We then apply a norm on \hat{X} to encourage the recovery of a smooth d.

For a single measurement vector, i.e., single pixel, the Fused LASSO (Tibshirani et al., 2005), which constrains the approximate derivative of x to be piecewise constant, and can be implemented by applying a the first-order differencing operator (D)

$${min}_{x} {‖ y - Ax ‖}_{2} + {lamda}_{1} {\vert x \vert}_{1} + {lamda}_{2} {\sum }_{i = 2}^{m} ( {x}_{1} - {x}_{i - 1} ) \\ {min}_{x} {‖ y - Ax ‖}_{2} + {lamda}_{1} {‖ x ‖}_{1} + {lamda}_{2} {‖ Dx ‖}_{1} \\ where D = \begin{matrix}0 & 0 & 0 & … \\ - 1 & 1 & 0 & \\ 0 & - 1 & 1 & \\ ⋮ & & & ⋱\end{matrix}$$

For our MMV-BP problem, the natural extension is to apply these operators to minimize ∥Dĉ∥₁ where row-cardinality ĉ=Σ₁^{p} Xₚ,.>0 is again the occurrence of each sequence in X. However, as threshold operations are non-convex, P we instead apply the differential operator D to the estimate Σ₁^{p} Xₚ,. (which we refer to as an L1 norm).

To encourage a sparse X, we compare the effect of three sparsening norms. Here, we compare a total variation constraint ∥X∥_{1,1}, an L2,1 norm that tolerates more small values of X, as well as a nuclear norm.

$${min}_{x} {‖ y - Ax ‖}_{2 , 1} + lamd {a}_{1} {‖ X ‖}_{1 , 1} + lamd {a}_{2} {‖ D {\sum }_{i = 1}^{p} X … , i ‖}_{1} \\ {min}_{x} {‖ y - Ax ‖}_{2 , 1} + lamd {a}_{1} {‖ X ‖}_{2 , 1} + lamd {a}_{2} {‖ D {\sum }_{i = 1}^{p} X … , i ‖}_{1} \\ {min}_{x} {‖ y - Ax ‖}_{2 , 1} + lamd {a}_{1} {‖ X ‖}_{*} + lamd {a}_{2} {‖ D {\sum }_{i = 1}^{p} X … , i ‖}_{1} \\ <$$

The motivation for the use of the nuclear norm is that, as the nonzeros in X arise from an i.i.d. sampling process on c, the approximation 1/p Σ₁^{p} X_{. . . ,i} is itself a rank-one estimate of c via the central limit theorem. Constraints on the rank of \hat{X} may therefore encourage a useful kind of sparsity, but are nonconvex—instead, we can apply the nuclear norm, which constrains the sum of singular values of \hat{X}. (Recht, 2007) For implementation, we seek solutions we implement these constraints in Basis Pursuit format, and optimize problems of the following form using the cvx package

$${min}_{x} {‖ \hat{X} ‖}_{1 , 1} s . t . {‖ Y - AX ‖}_{2 , 2} < sigma , {‖ D {\sum }_{i = 1}^{p} X … , i ‖}_{1} < rho$$

In conclusion, copy number variation arising due to segmental duplications or deletions can be recovered by enforcing smoothness in the recovery matrix, as a fused LASSO problem using a differencing operator. (See FIG. 15 and FIG. 16)

Applying MIXSEQ to Sequences Generated from FISSEQ

(a) Introduction

The MIXSEQ approach relies on a tight integration of molecular biology, i.e. sequencing and math (compressed sensing), to enable the demixing of superimposed DNA sequences. Generally, we are interested in counting elements of DNA, rather than de novo sequencing. The design of primers for reverse transcriptase, amplification and sequencing all happen in concert and play an important role. The design of primers determines three critical factors: (1) which mRNAs will be sampled by the sequencing process; (2) the exact sequences that will be read via FISSEQ (target sequences); and (3) the contents of the sequence dictionary that will be used for demixing.

In all cases below, the result is a cDNA of some kind that contains a target sequence that will be amplified. These target sequences are equivalent to the sequences that would normally arise during de novo sequencing—the only difference is that they are known advance.

Target sequences are intentionally chosen so that they are as different as possible from one another, according to a variety of metrics. In addition, these dictionary elements may be chosen to conform, or nearly conform, to a known error-correcting code such as a Hamming code or Levenshtein code. (See, e.g., Buschmann and Bystrykh, 2013). This choice defines the sequence dictionary and is critical to our technique.

(b) Experimental Outline
1. Design primers, and make a dictionary of expected target sequences
2. Process tissue for FISSEQ
3. Perform reverse transcription (RT)
4. Amplify cDNA
5. Run sequencing reaction
6. Identify an interesting set of pixels (“measurement matrix”)
7. Unmix sequence within the measurement matrix using MIXSEQ

(c) Experimental Details

1) Design primers and sequencing approach—This is a critical step and is required to happen first. Details are folded into the text below.

2) Process tissue for FISSEQ—Although this process may vary, generally the steps include:

a) Lightly fixing the tissue;
b) Digesting away DNA;
c) Digesting away protein; and
d) Fixing the RNA via cross-linking (possibly during fixation, possibly after reverse transcription).

3) Perform reverse transcription—A critical feature of the MIXSEQ technique is that it allows RT/amplification to generate rolonies at high densities, such that they overlap optically and/or physically.

The methods used to do this can vary significantly e.g., changing primers, changing RT conditions, using PADLOCK probes, etc. However, the end result is a population of rolonies that arise at such high density that they would not normally provide useful sequencing information. The assumption here is that the techniques described herein give rise to this superposition.
a) In the standard approach, primers are designed such that the dictionary of nucleotides that will be sequenced as the endogenous RNA are immediately downstream of the primer binding site. Here, the set of target sequences is typically defined by the nucleotides immediately downstream of the RT primer binding site. Although these target sequences are in fact drawn from the genome, the specific set of target sequences will end up as a physical unit later when the sequencing primers are designed.
b) In another instantiation, RT primers can include a transcript-specific barcode as part of their sequence. This transcript-specific barcode is independent of the mRNA binding sequence and is only sequenced if the 3′ end of the primer successfully bound to and amplified a portion of the endogenous mRNA. This is useful because it allows relatively similar mRNAs, for instance, homologues, to be identified via barcodes that are very dissimilar. It also generates a common signal from RT primers targeting the same mRNA in different places i.e., with different mRNA binding sequences, that have the same FISSEQ signal after amplification.
The barcodes designed here can either be random (arbitrary, but gene-specific) or can be designed carefully to avoid overlap with barcodes corresponding to other genes. In this case, they may be considered standard error-correcting codes.

4) Amplify cDNA—The cDNA containing our target sequence is then amplified, typically using rolling circle amplification (RCA). The result of this amplification is a rolony.
a) An amplification primer is designed to enable RCA—this is called the RCA primer.
   i) The RCA primer uses a padlock probe. In this case, the primer binds to the cDNA in two places, generating a loop structure that defines the sequence to be amplified. Typically, the amplified sequence may include: (1) a portion of the RT primer, (2) portion of the mRNA, and (3) the entirety of the RCA primer. Additionally, in various instantiations, the target sequence can be part of either: (1) the RT primer, (2) the targeted mRNA, or (3) the RCA primer. Next to the target sequence there will also be a binding site for the sequencing primer.
   ii) The original FISSEQ method utilizes a slightly different process, using circLigase.
b) A displacing polymerase is used to perform rolling circle amplification using the RCA primer. This amplifies the target sequence along with some other sequences that are part of the RCA primer, targeted cDNA, etc.

5) Run FISSEQ reaction to sequence the target sequence—the FISSEQ reaction uses either Illumina or Solid Sequencing chemistry to generate a fluorescent signal that corresponds to the target sequence.
a) A sequencing primer is designed to target the target sequence, and this primer binds upstream of the target sequence. Note that each targeted mRNA molecule has been amplified such that it has hundreds-thousands of target sites.
b) Each base in the target sequence is sequenced using sequential chemistry for each base. For each position, this roughly involves:
   i) A mix of fluorescent nucleotides is applied to the sample along with a polymerase. One fluorescent nucleotide of the appropriate base is incorporated at the first position.
      Note: For Illumina sequencing, the color of this fluorescence signals the identity of the nucleotide directly. For SOLiD sequencing, mapping between color and sequence position requires minor additional steps. Additionally, novel methods of sequencing that intentionally complicate the relationship between color and sequence—but are useful because they may make the demixing problem more tractable—may also be practiced. Such methods include, for instance, sequencing both ends of a barcode, simultaneously generating a mixed signal for even a single barcode, among others.
   ii) Excess nucleotides are washed off.
   iii) Fluorescence arising from rolonies is imaged using multi-color microscopy. This can be imaged using a confocal microscope or other microscope.
      The expectation is that this signal will be mixed at many or all pixels. That is, instead of seeing a single color corresponding to one base (and indeed a “spot” corresponding to one rolony) we will see multiple colors from multiple rolonies, and may not be able to easily distinguish rolony borders.
      In computational language, for traditional FISSEQ we expect to see a noisy version of the pure signals [1,0,0, 0] [0,1,0,0] [0,0,1,0] or [0,0,0,1] corresponding G, A, T, or C.
      For MIXSEQ, we expect to see mixtures at each base, e.g. [0.33 0.33 0.33 0] which would correspond to a mixture of three rolonies encoding G+A+T but not C.
      Many parameters of the microscope determine the form of this signal, including (1) the number of pixels imaged within as given field of view, (2) the care taken to reduce noise, (3) the number of available color channels, and (4) the overall level of magnification.
   iv) The fluorescent signal is terminated, and sequencing is repeated for the next position.

6) The intensities from multiple pixels are consolidated into a coherent measurement matrix—The measurements made during sequencing arise as a set of multi-color snapshots, with one snapshot for each base position. Each snapshot may be 2D or 3D, depending on whether a full volume is being imaged.
a) The measurements are consolidated made during sequencing into a measurement matrix—this is basically a re-organization of the original measurement.
b) Select a relevant subset of pixels for demixing—In some cases, we will demix a single pixel at a time. In other cases, we will identify subsets of pixels (such as a 4×4 square, or all pixels associated with a given cell) and group them together for demixing. This grouping procedure may either be by averaging, or by using those pixels to define a subproblem that is easier to solve than the full measurement matrix. Whatever subselection is made here, we will continue to call this set of pixels as a measurement matrix.
c) Identify the barcode sequences that give rise to these pixel-signals via demixing—This is the core algorithm at work for FISSEQ neuronal barcoding. These steps may be applied on the microscopy/sequencing system, or the raw data may be transferred to another system.
   There are several alternative approaches:
   i) Using the dictionary of reference sequences, the small number of target sequences that give rise to the measurement matrix are identified. We term the output the sparse solution.
      Single-pixel: what (small) set of target sequences gives rise to the mixture observed in this measurement matrix?
      Multi-pixel: what (small) set of target sequences is distributed across the pixels in this measurement matrix?
      For the single-pixel problem, we usually use the assumption of simplicity or sparsity (that only a small number of possible target sequences is actually present in the signal) to find the correct solution. We typically quantify that sparsity using the L0, L1, L2, or other vector norm or matrix pseudo-norm.
      For the multi-pixel solution, solutions that are “sparse” may be found in one or both of these ways: (a) sparse in the sense that only a few possible target sequences are actually present in the pixel signal, (b) smooth in the sense that pixels are relatively homogenous between neighboring pixels or groups of pixels, or (c) constrained by additional information such as the knowledge that some target sequences are likely to covary within a given mixed sequencing signal, or that the overall prevalence of some target sequences is likely to covary across a set of mixed sequencing signals.
      The actual algorithms used here can be variants of matching pursuit, basis pursuit, approximate message passing, belief propagation, a neural network, or a convex or non-convex solver of any kind. For example, LASSO, basis pursuit, matching pursuit, and neural networks are each algorithms (or classes of related algorithms) that can effectively recover sparse solutions to the sequence demixing problem.
   ii) The sparse solution is applied to the biological question—In some cases, the solution to the biological problem is found by simply counting the number of pixels that contain any given target sequence. For example, in transcriptome sequencing, there may be a specific interest in the number of transcripts for each gene. In other cases, such as connectome sequencing, there may be less interest in counting the number of rolonies and instead an interest in precisely defining the location of a given rolony. For example, one goal may be to identify which barcodes or DNA sequences are associated with a given cell or morphological feature of a cell.

Compositions of matter that give rise to mixed sequencing signals. In the general context of oligonucleotide sequencing, there are many compositions that give rise to mixed sequencing signals. In general, mixed sequencing signals arise whenever two or more unique target sequences are amplified and recovered during sequencing within one pixel or a set of contiguous pixels.

For example, a mixed sequencing signal may arise when two rolonies are amplified and sequenced in close proximity to one another, with one rolony arising from a PLA reaction associated with an oligonucleotide, and the second rolony arising by association with a protein (FIG. 17A).

For example, a mixed sequencing signal may arise when multiple subsequences within a single oligonucleotide are targeted by hybridization of an RNAScope-style set of hybridization probes (FIG. 17B, top), by a set of Stellaris-style hybridization probes (FIG. 17B, middle), or by the Proximity Ligation Assay (FIG. 17B, bottom). In each case, the mixed sequencing signal arises from the sequencing of hybridization probes or amplicons derived from hybridization probes that are associated with multiple distinct target molecules. Here we consider that each hybridization probe (or amplicon derived from a hybridization probe) associated with one target molecule such as an mRNA shares a common target sequence, but that different target sequences are associated with different molecules.

For example, a mixed sequencing signal may arise when multiple subsequences within a single oligonucleotide are targeted by hybridization of an RNAScope-style set of hybridization probes (FIG. 17B, top), by a set of Stellaris-style hybridization probes (FIG. 17B, middle), or by the Proximity Ligation Assay (FIG. 17B, bottom). In each case, the mixed sequencing signal arises from the simultaneous sequencing of distinct hybridization probes or amplicons derived from hybridization probes that are associated with different regions of a single target molecule. Here, we consider that each hybridization probe (or amplicon derived from a hybridization probe) has a distinct target sequence.

A mixed sequencing signal may arise when a single amplicon such as a rolony is made into a double-stranded molecule, and then sequenced in two directions simultaneously. When sequenced in one direction (FIG. 17A) the sequencing signal is not mixed. When sequenced in two locations at the same time (FIG. 18B) this gives rise to a mixed sequencing signal.

In a modification of traditional PLA, amplification of one rolony (red) may be dependent on proximity to a second rolony (green). When only one rolony is amplified, this gives rise to an (unmixed) sequencing signal (FIG. 19A). However, when two such rolonies are amplified, with each rolony carrying a different target sequence, this gives rise to a mixed sequencing signal (FIG. 19B).

Under standard sequencing, a target sequence (for example, GTACGTCCGAC) has a corresponding sequence matrix that is not mixed. (FIG. 20A) However, under convolutional sequencing, we may enable a portion of the sequencing molecules within an amplicon to pass through one step of sequencing and generate a signal from the second step. (FIG. 20B). This gives rise to a different sequencing matrix, but which can be deconvolved into the original sequence matrix as necessary. (FIG. 20B). When multiple target sequences are sequenced within the same pixel or set of pixels, these convolved sequencing matrices may result in a mixed sequencing signal, which can be subsequently demixed by our method. The example shown here for a single pixel, but remarkably may also be applied across many pixels.

Under some circumstances, we may use a neural network to demix a set of mixed sequencing signals. One possible architecture for this neural network is shown in FIG. 21.

Unless otherwise defined, all technical and scientific terms used herein have the same meaning as commonly understood by a person of ordinary skill in the art to which this invention belongs.

As used herein, and unless stated otherwise or required otherwise by context, each of the following terms shall have the definition set forth below.

As used herein, the term “nucleotide” refers to a nucleotide of any length, which can be DNA or RNA, can be linear, circular or branched and can be either single-stranded or double-stranded.

As used herein, the term “sequence” refers to the sequence information encoded by a nucleotide molecule.

As used herein, the term “gene” includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins and locus control regions.

As used herein, the term “target sequence” refers to the sequence of interest which is selected, amplified, and revealed via the sequencing operation. This sequence is represented in a traditional format via the oligonucleotide bases (e.g. G, T, A, C, and U) or in a similar textual format.

As used herein, the term “sequence matrix” of a given oligonucleotide sequence refers to a representation the sequence content of an oligonucleotide in a matrix format that is appropriate for a given sequencing methodology. For example, a sequence matrix might be represented as a matrix where each row and column represent the fluorescent intensity associated with a given sequencing step (for each row), and each channel of the microscopy image (for each column) For example, using 4-color Illumina Hiseq chemistry this representation has an intuitive form: a given target sequence may be represented by the fluorescent signal expected during sequencing: that is, in numerical matrix where one dimension (e.g. rows) represents a position along the oligonucleotide sequence, and another dimension (e.g. columns) represent the possible nucleotide bases (G,T,A, or C). For Illumina Hiseq chemistry, the nucleotides might be represented as G=[1 0 0 0], T=[0 1 0 0], A=[0 0 1 0], C=[0 0 0 1] with appropriate ordering of fluorescent channels, and the sequence “GTAC” is represented as [[1 0 0 0], [0 1 0 0], [0 0 1 0], [0 0 0 1]]. The same target sequence may have differing sequence matrix representations depending on the chemistry used during sequencing. For example, Illumina NextSeq chemistry uses only two colors, and the sequence G=[1 0], T=[0 1], A=[1 1], C=[0 0]. As another example, the SOLiD Sequencing method does not have a one-to-one relationship between each sequencing reaction (i.e. each sequencing image) and a given position in the target sequence, but can still be represented as an appropriate sequence matrix. In this case, a representation of an oligonucleotide sequence appropriate for SOLiD sequencing might represent a sequence as a series of ligation steps in one dimension (for example, rows) and fluorescent output channels (for example, columns). In addition, the sequence matrix representation may incorporate information about bleed-through between microscopy channels or expected intensity associated with each microscopy channel and may thus represent the expected output of a specific microscope or microscope configuration. In addition to these variations of sequencing chemistry, a sequence matrix may be reordered without losing or changing its content—for instance, by transposition, or transformation into a vector by concatenating the rows or columns of a sequence matrix.

As used herein, the term “sequence vector” refers to a reshaping a sequence matrix into a vector, either by concatenating the rows or columns of a sequence matrix or through some other reordering.

As used herein, the term “sequencing signal” refers to the signal arising from the sequencing reaction (i.e., the fluorescent output) for a single pixel or collection of pixels. The sequencing signal can be represented in “matrix format”, with each row corresponding to a position along the linear RNA/DNA molecule, and each column corresponding to a different channel arising from fluorescence. For example, using 4-color Illumina sequencing (HiSeq), each column would correspond to the fluorescent signal associated with one nucleotide base (G,T,A, or C). For instance, a blue fluorescent output signal indicates a CTP was incorporated into the strand being synthesized by the sequencing reaction. Similar correspondences can be made for sequencing methods that do not spectrally separate each nucleotide base in a trivial manner (such as two-color sequencing in NextSeq, or the more complex color scheme associated with SOLiD Sequencing). In general, the “sequencing signal” can be considered as a matrix or vector representation of the raw signal arising from the sequencing reaction, either directly or after some appropriate mathematical transformation. In addition, the “sequencing signal” may be transformed from a matrix as described above into a “sequence vector”.

As used herein, the term “sequence dictionary” refers to the set of reference sequences which may be present in a particular biological sample. The membership of this set is determined jointly by the biological sample, and the processes used to select and amplify the DNA or RNA (cDNA). For instance, when MIXSEQ is applied to a genome sequencing, in which a set of sequences derived from the genome are sequenced to a length of 250 base-pairs, the dictionary may be considered to be the set of all possible unique sequences of length 250 that are contained within the genome. As further explained below, the sequence dictionary may not be known in advance, or may be partially known in advance, with membership of the set of reference sequences determined as an application of additional relational information about, inter alia, the mixed sequencing signals, the pixels, known reference sequences, and/or target sequences.

The set of reference sequences contained within a sequence dictionary is determined by factors such as the primers used for reverse transcription, circularization, and sequencing. Each reference sequence in a sequence dictionary may be represented in standard text form (for example, GTAC) or in the form of a sequence matrix appropriate for a given sequencing methodology.

As used herein, the term “mixed sequencing signal” refers to a sequence vector which represents sequencing information in which information from two or more individual sequences is superimposed: For example, the sequencing signal corresponding to a single pixel may generate a “mixed sequencing signal” if the field of view associated with that pixel contains two unique molecules with different sequences. As another example, the sequencing signal corresponding to a single pixel may generate a “mixed sequencing signal” if two isolated subsequences on a given oligonucleotide are sequenced at the same time.

As used herein, a reference sequence, reference sequence vector, or reference sequence matrix is considered “representative of” a mixed sequencing signal, mixed sequence matrix, or mixed sequence vector, where the reference sequence, reference sequence vector, or reference sequence matrix are sufficient to explain the variability of the mixed sequencing signal, mixed sequence matrix, or mixed sequence vector with an error less than 90%, 75%, 50%, 25%, 10%, 5% or less.

As used herein, the term “next generation sequencing” or “NGS” refers to any modern high-throughput sequencing technology. NGS includes, but is not limited to, sequencing technologies such as Illumina (Solexa) sequencing and SOLiD sequencing. A major advantage of NGS over previous sequencing technologies is the ability to perform massively parallel sequencing, in which many sequences are read in parallel but are not mixed. The invention herein provides a method, referred to herein as “MIXSEQ,” which allows for deconvolution of previously unusable, mixed data generated by massively parallel sequencing, MIXSEQ is particularly useful for in-situ sequencing methods such as FISSEQ.

As used herein, sequencing in parallel includes, at least, simultaneously sequencing regions originating from multiple distinct oligonucleotides, or simultaneously sequencing multiple regions of an oligonucleotide.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

### BRIEF DESCRIPTION OF THE DRAWINGS

FIG. 1: Demixing the grocery list—utilizing a pseudo linear algebra framework to resolve a mixed signal. With appropriate changes, the same principle can be used to resolve mixed sequencing signals.

FIG. 2: Comparison of traditional and MIXSEQ-enabled sequencing workflows. Traditional sequencing workflows rely on base-calling of individual pixels or groups of pixels containing unambiguous sequencing signals. In contrast, a MIXSEQ-enabled workflow generates ambiguously mixed sequencing signals that can be recovered by comparison to a database or dictionary of sequences (which is either known or unknown before the experiment).

FIG. 3: Representation of a sequence matrix or sequencing vector. The sequencing signal from a single pixel can be represented as a matrix or vector of pixel intensities across multiple channels and nucleotide positions.

FIG. 4: The sequencing problem redefined as a linear algebra problem. Once a mixed sequencing signal is recovered, representation as a sequencing vector allows for the demixing problem to be framed as a (typically underdetermined) linear algebra problem. See also FIG. 5 for a mathematically explicit representation of this problem.

FIG. 5: Alternative schematic depicting the MIXSEQ process for determining individual sequences from mixed sequencing images.

FIG. 6: Recovery of mixed sequences with different number of components (k) from a dictionary of size 10,000 random barcodes. Here we show the recovery error associated with the coefficient's matrix X. For example, it is possible to demix 8 overlapping sequencing signals as long as the total sequence length is greater than approximately 40.

FIG. 7: The phase transition for solvability of a compressed sensing problem. This problem was solved using Orthogonal Matching Pursuit, using a random Gaussian dictionary and log-normal loadings. n=1000.

FIG. 8: Comparison of different sensing dictionaries.

FIG. 9: Effect of Dictionary Size on Recovery Threshold—Here we show the demultiplex threshold (i.e. k-sparsity that allows successful demixing) for barcode dictionaries of different sizes. Mixed signals were generated using unit loadings, and demixed using Orthogonal Matching Pursuit. For a dictionary size approximately the size of the mouse genome (10,000 sequences), it is feasible to demix up to 32 mixed sequences after sequencing approximately 150 bases.

FIG. 10: Resistance to noise—Compressive sensing for sequencing under high noise. Using a dictionary of 10,000 random barcodes with unit loadings, we modeled the recovery of each mixture using non-negative Orthogonal Matching Pursuit (OMP) under additive Gaussian noise. Under noise that matches current Illumina sequencing technology (a Q-score of approximately 40), we observe robust demixing of approximately 8 overlapping DNA sequences as long as 250 bases are sequenced.

FIG. 11: SNP detection. (Left) The magnitude elements of A, conditioned on bases known to be correct (CX) and carry mutation (SNP). (Right) ROC analysis of SNP detection for varying thresholds on the range metric. The overall AUC (0.91) suggests that it 90% of SNPs can be recovered from a mixed sample.

FIG. 12: NMF recovery of 10 10-mer barcodes from 1000 simulated colonies (with random spacing). When applied to a simulated mixture of sequencing signals, both NMF and ICA are capable of recovering the mixed sequence information. In this example, non-negative ICA is more effective than NMF at recovering the exact set of mixed sequences.

FIG. 13A: Recovery with a known dictionary—Overview. In this biological example, many cells are labeled with unique barcode, with the goal being to recover overlapping barcodes that may arise in each pixel.

FIG. 13B: Recovery with a known dictionary—Grouping Mask. Isolating pixels with sequencing signals of relatively large magnitude reduces the scale of the recovery problem. Pixels with similar sequencing signals can be grouped to further simplify analysis.

FIG. 13C: Recovery with a known dictionary—Sequencing Images. Raw sequencing images are shown across four imaging channels (corresponding to columns labeled G,T,A, and C) for five positions (each corresponding to a row).

FIG. 13D: Recovery with a known dictionary—Group LASSO. Given a grouping matrix G, we find

min x∥Y−AX∥_{2,1} s.t. ∥X∥<beta s.t.∥XG∥<lambda.

We then show recovered loadings for each dictionary element which correspond to recovered cells.

FIG. 14A: Unknown dictionaries—Overview. Example biological image showing neurons expressing a mixture of barcodes, to be recovered without knowing the dictionary of possible barcode sequences.

FIG. 14B: Unknown dictionaries—Recovered Barcodes. Following application of NMF to a mixture of sequencing signals (top left panel), we recover barcodes that match the known ground truth (top right panel). Recovered barcode are uncorrelated in their loading onto individual pixels (bottom left panel), as well as in sequence (bottom middle panel). The appropriate number of recovered barcodes can be identified by analysis of the L-curve, or by cross-validation (bottom right panel.

FIG. 14C: Unknown dictionaries—Sequencing Images. Raw sequencing data used for recovery, shown for four sequencing channels (corresponding to bases G, T, A, and C) for two sequential base positions. Lower panel shows zoomed inset.

FIG. 14D: Unknown dictionaries—Recovered Loadings. The pixel loadings of four barcodes are shown for a subset of the sequenced pixels.

FIG. 15A: Results of multi-task LASSO for estimation of Copy number Variation (i.e. CNV). The problem was modeled as:

$${min}_{x} {‖ \hat{X} ‖}_{1 , 1} s . t . {‖ Y - AX ‖}_{2 , 2} < sigma , {‖ D {\sum }_{i = 1}^{p} X … , i ‖}_{1} < rho$$

Copy number variation along the chromosome was modeled as an alternating stairstep. Due to Poisson sampling of individual sequences along the chromosome, recoverable estimates of CNV are noisy (\hat{X}, green line), and must be smoothed. The regularized estimate (X, magenta line) is identical to the ground truth.

FIG. 15B: Row sum of coefficients, i.e., Σₚ \hat{X}

FIG. 15C: The first derivative of the summed coefficients, i.e., D*Σₚ \hat{X}.

FIG. 15D: The second derivative of the summed coefficients, i.e., D².Σₚ \hat{X}.

FIG. 16A: Additional non-limiting examples of sequencing methods that may give rise to mixed sequencing images which MIXSEQ can be applied to—Protein/RNA localization, e.g. when multiple subsequences within a single oligonucleotide are targeted by hybridization of an RNAScope-style set of hybridization probes;

FIG. 16B: RNAscope-style sequencing, e.g. RNAScope-style set of hybridization probes (top), a set of Stellaris-style hybridization probes (middle), or Proximity Ligation Assays (bottom). In this example, overlapping sequences arise from the sequencing of molecules that are bound to a target mRNA, and are either directly hybridized to the target mRNA or hybridized with one or more intervening oligonucleotides that are themselves hybridized to a target mRNA. In some instantiations, the sequencing target is amplified. In this example, a plurality of the sequenced oligos arising from a single mRNA share a common sequence that is revealed during the sequencing reaction—however, spatial proximity to other mRNAs results in overlapping signals.

FIG. 16C: Intramolecular sequence barcoding or intramolecular barcoding in conjunction with sequencing. In this example, each hybridization event onto a target mRNA may carry a sequence signature that is distinct from sequences associated with other hybridization events on the same target mRNA. As these hybridization events are in tight spatial proximity, the resulting sequencing signal is a mixture of several underlying sequences.

FIG. 17A: Traditional rolony sequencing, in one direction, yielding a standard, unmixed result.

FIG. 17B: Simultaneous Bidirectional rolony sequencing, yielding a mixed sequencing result. In this example, a single rolony is read out in a bidirectional fashion, either using a standard rolony or after double-stranding. The resulting sequencing signal is thus composed of two unique signals from the same rolony or amplicon.

FIG. 18A: Comparison of proximity-dependent amplification of one rolony using Proximity Ligation Assay followed by in-situ sequencing. When only one rolony is amplified, this yields a standard, unmixed result.

FIG. 18B: Proximity Ligation Assays (e.g., as shown in FIG. 17B, bottom) result in spatial proximity of amplicons to other mRNAs, resulting in overlapping signals. When two rolonies are amplified under such conditions, each carrying a different target sequence, this yields a mixed sequencing result.

FIG. 19: Convolutional sequencing e.g., use of variant sequencing chemistry which utilizes partial termination at each sequencing step resulting in mixed sequencing images that can be both deconvolved and demixed using MIXSEQ.

FIG. 19A: Readout of standard sequencing chemistry is depicted, with 0% pass-through.

FIG. 19B: Readout of non-terminating chemistry at 50% pass-through. Convolutional sequencing may enable a portion of the sequencing molecules within an amplicon to pass through one step of sequencing and generate a signal from the second step. This gives rise to a different sequence matrix, but which can be deconvolved into the original sequence matrix as necessary FIG. 19C: Readout of non-terminating chemistry at 50% pass-through, mixture of two sequences. These convolved sequencing matrices may result in a mixed sequencing signal, which can be subsequently demixed by our method.

FIG. 20: Example architecture of a neural network that allows dictionary learning and recovery from mixed sequencing signals from an image. Many variant architectures are possible, but this example relies on a series of convolutions to generate a bottleneck layer (D) that represents the expression of individual barcodes across multiple pixels.

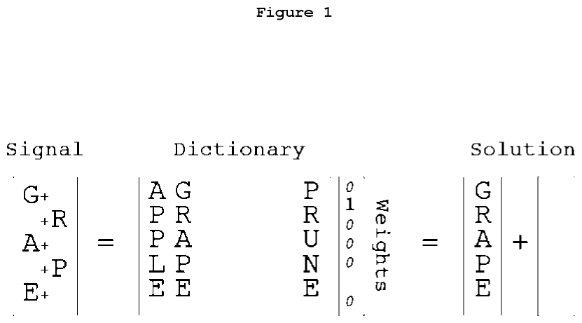

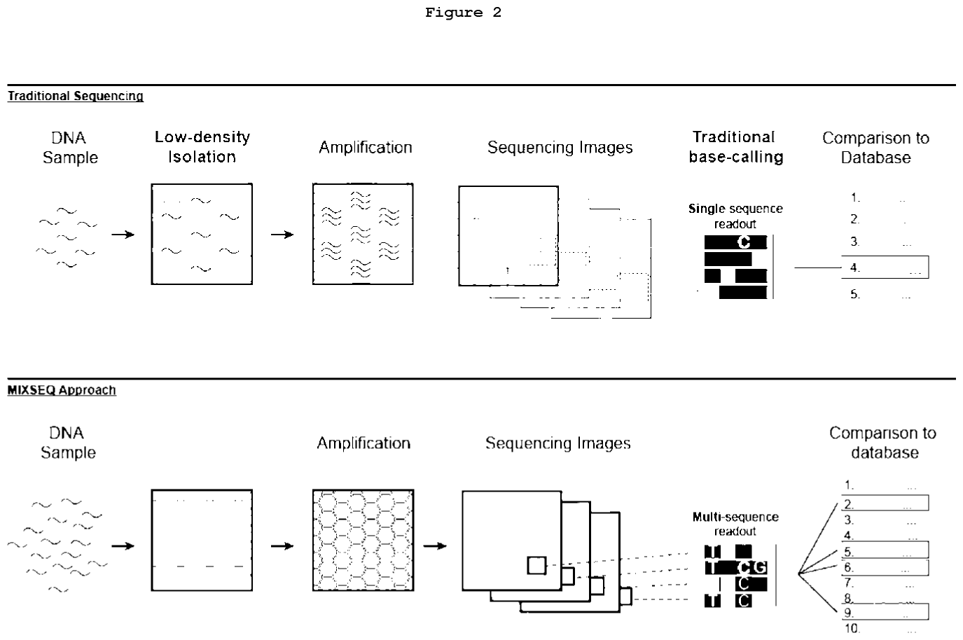

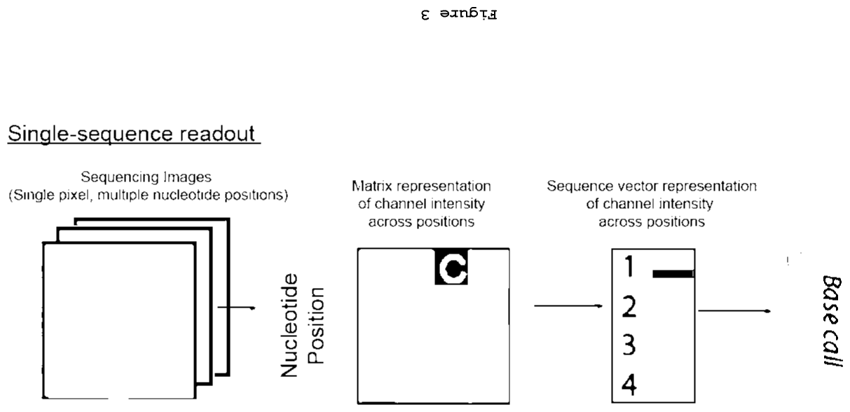

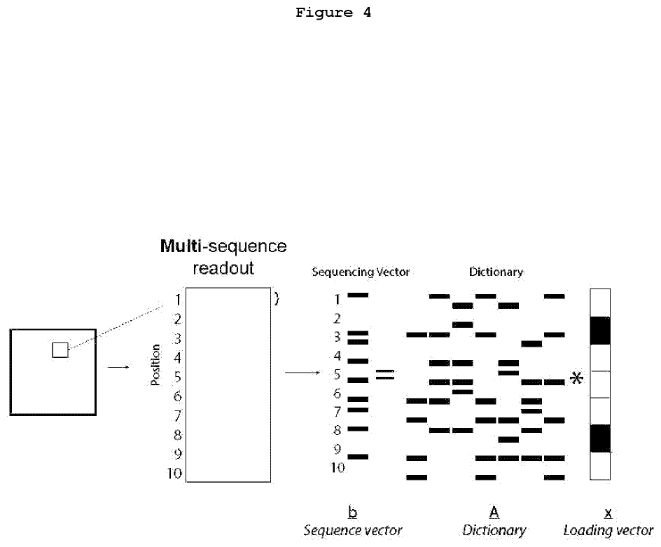

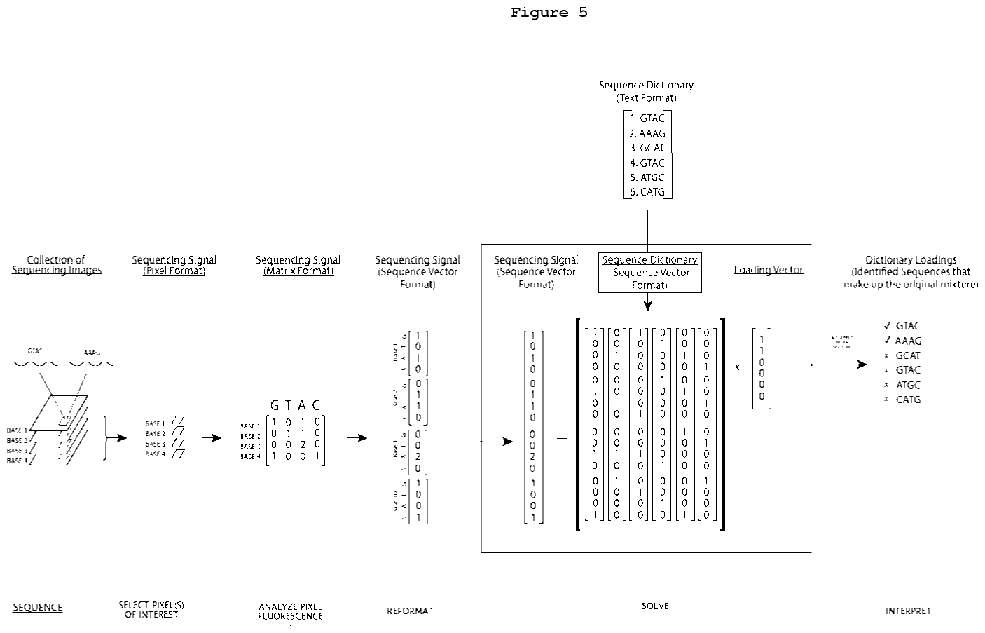

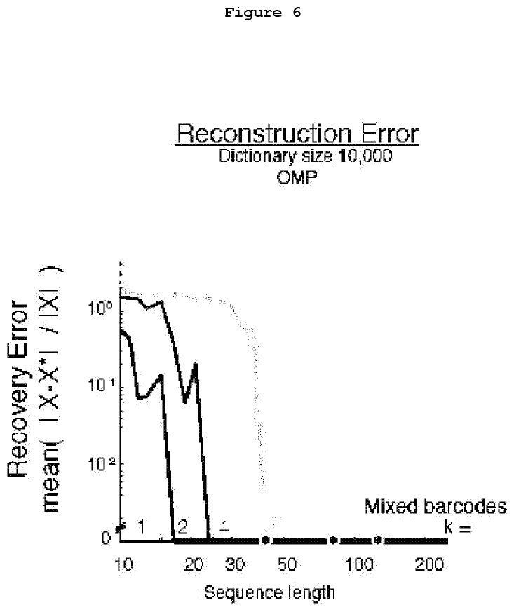

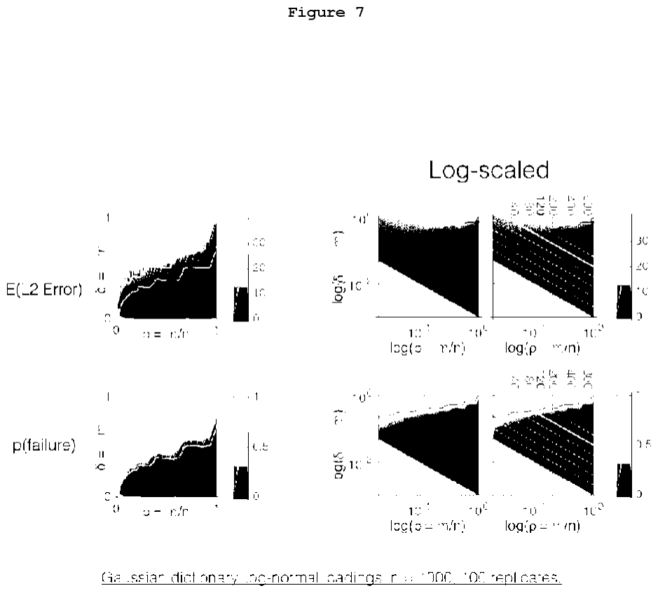

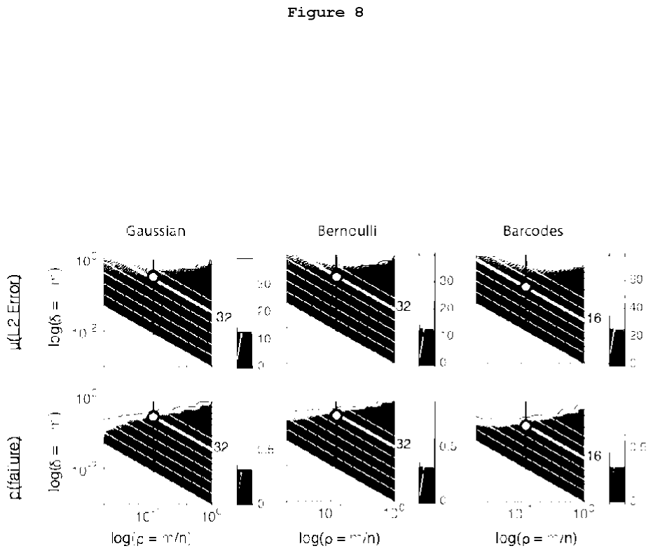

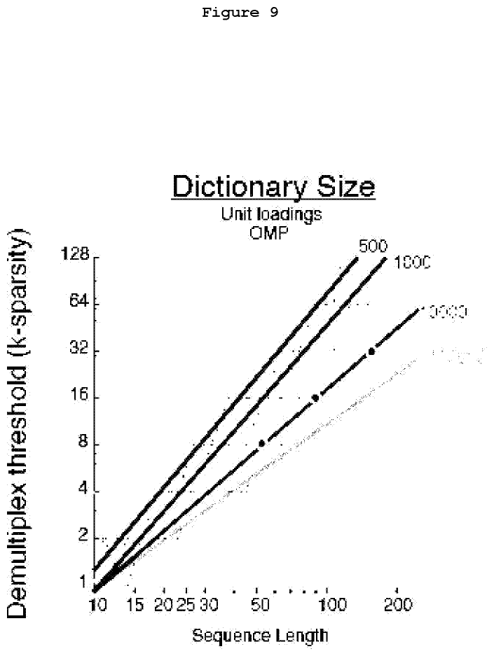

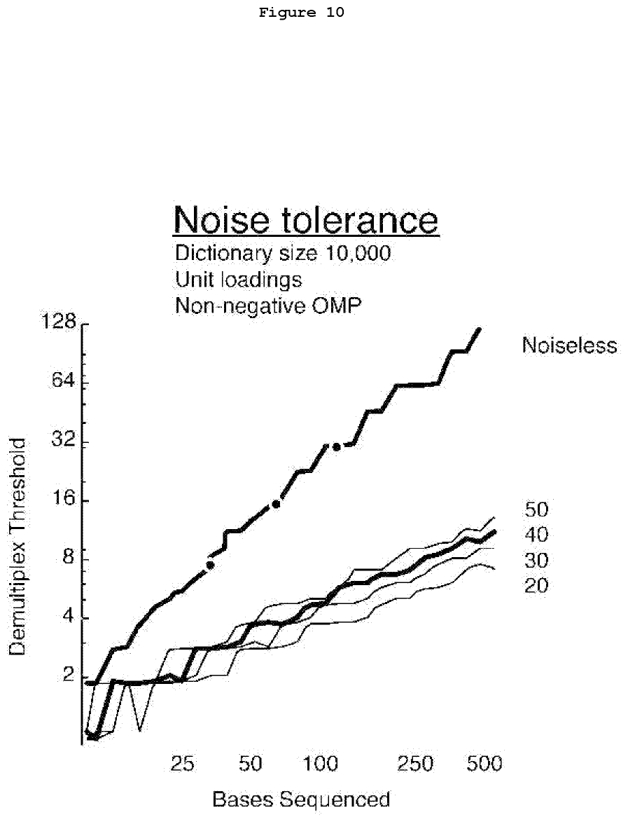

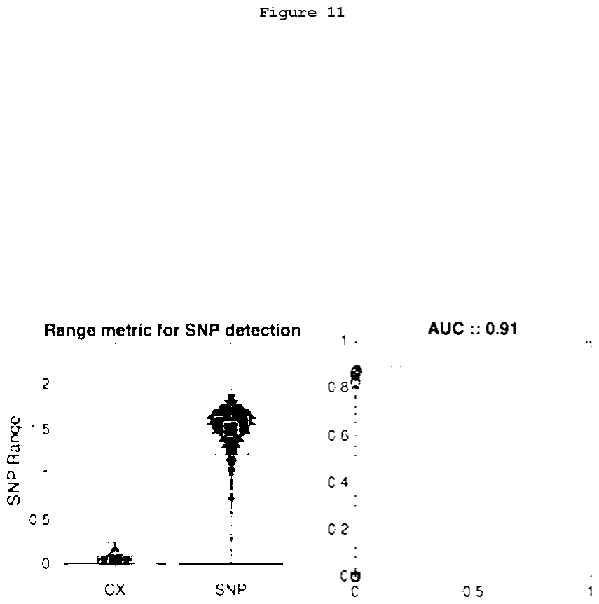

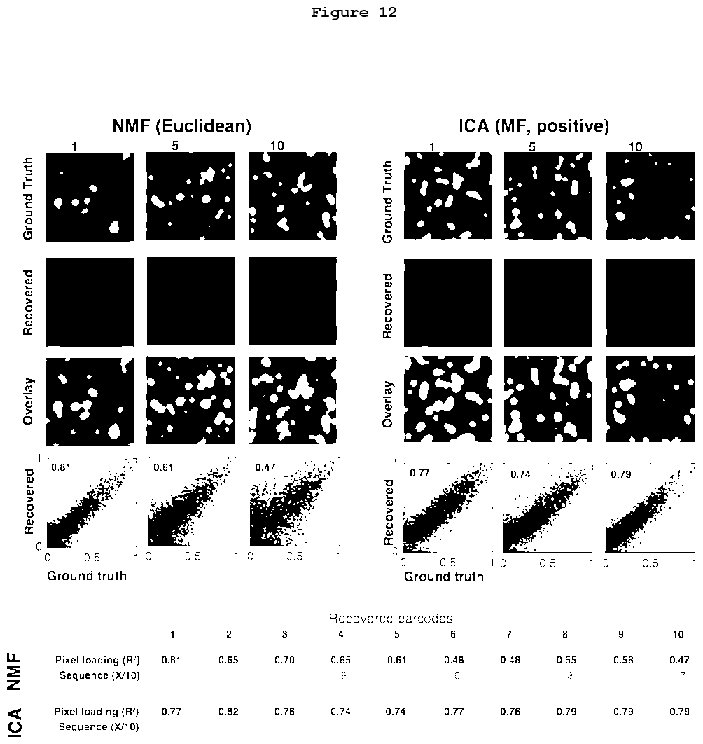

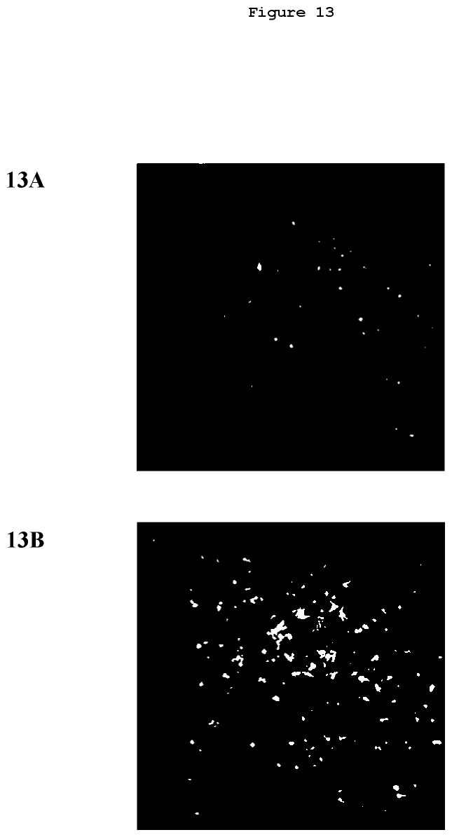

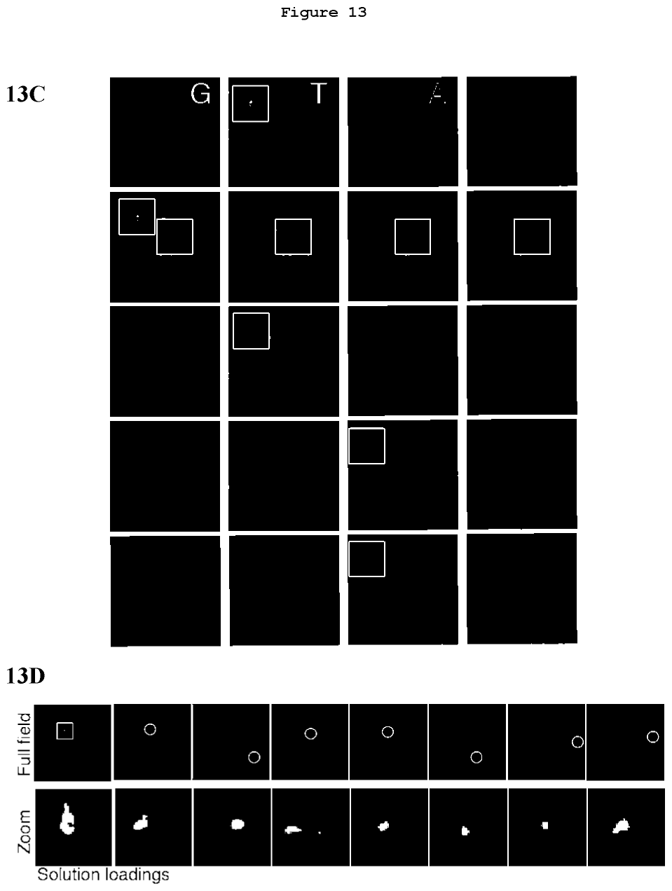

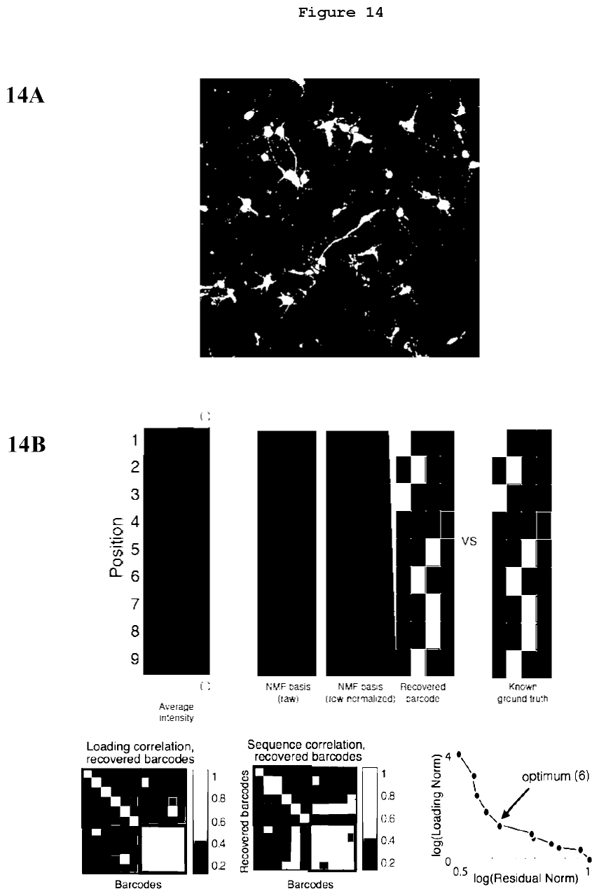

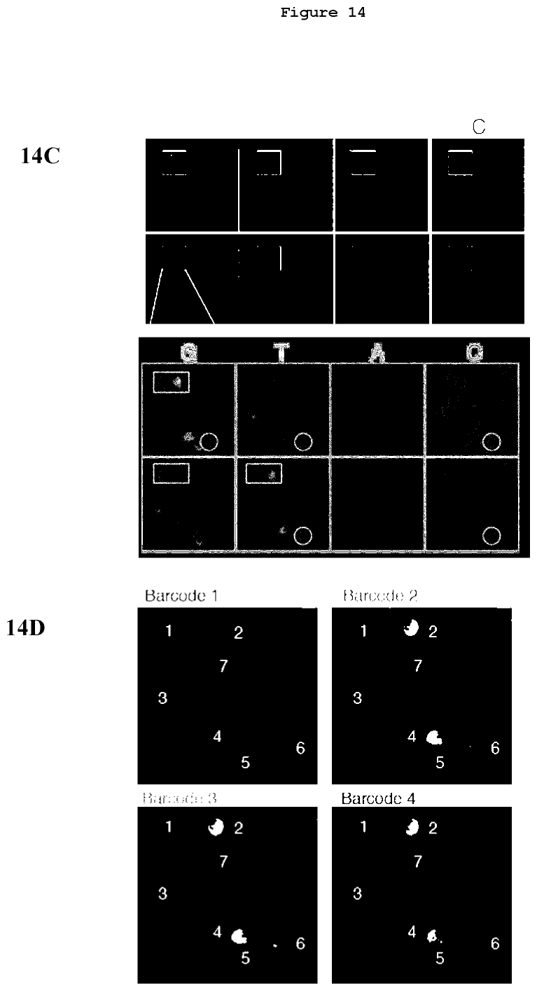

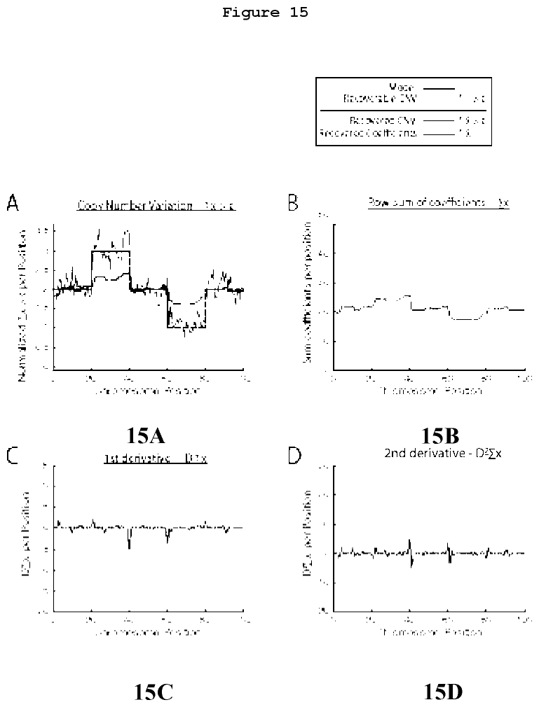

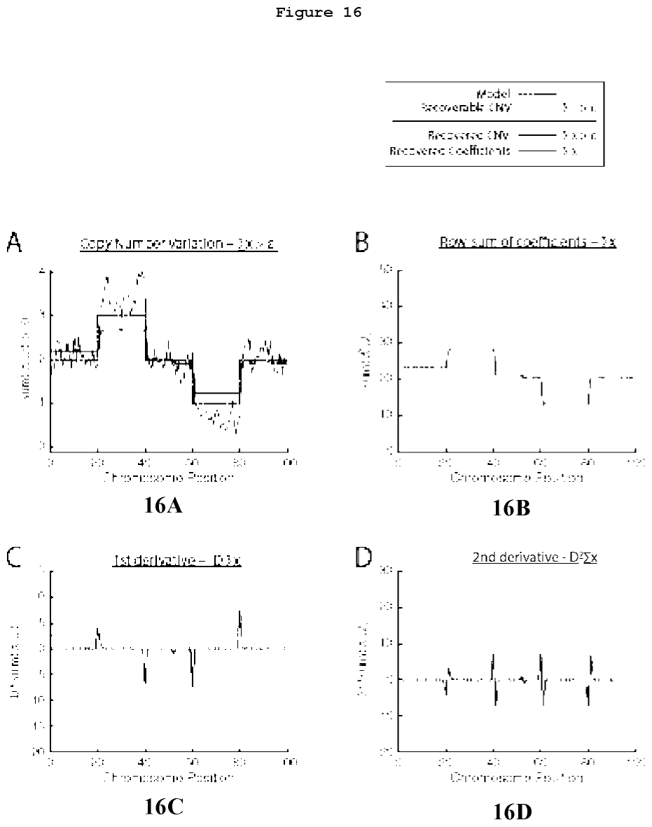

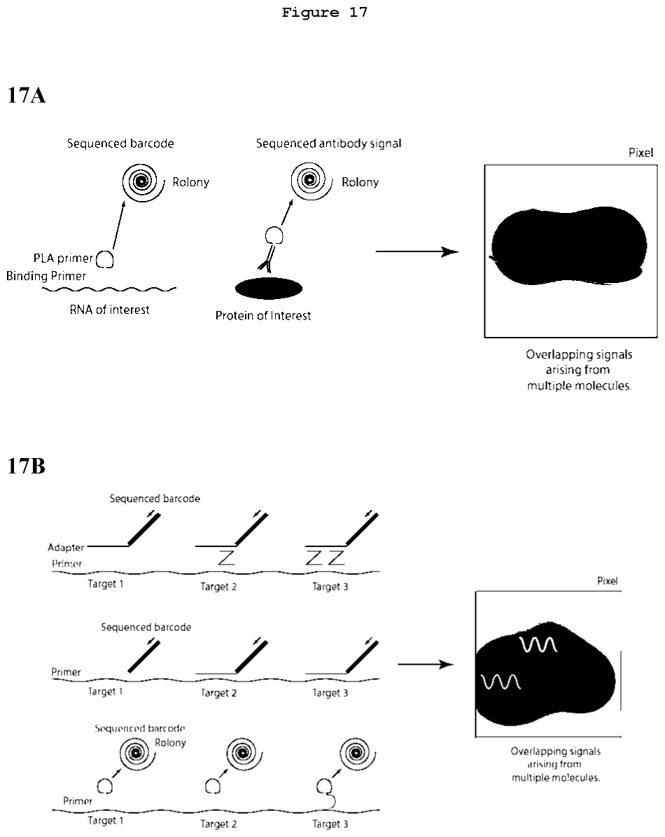

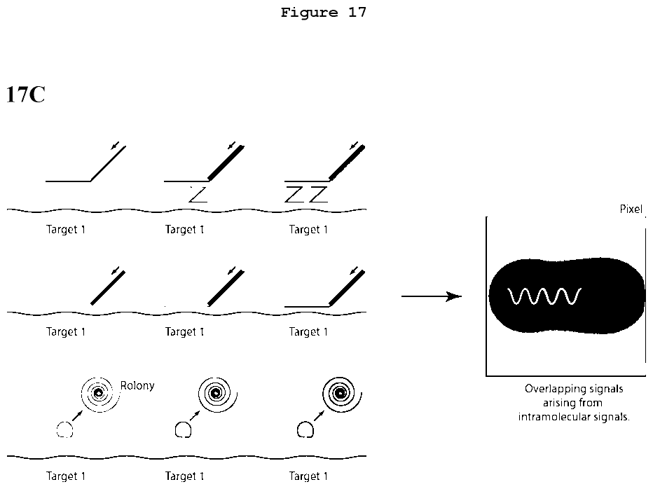

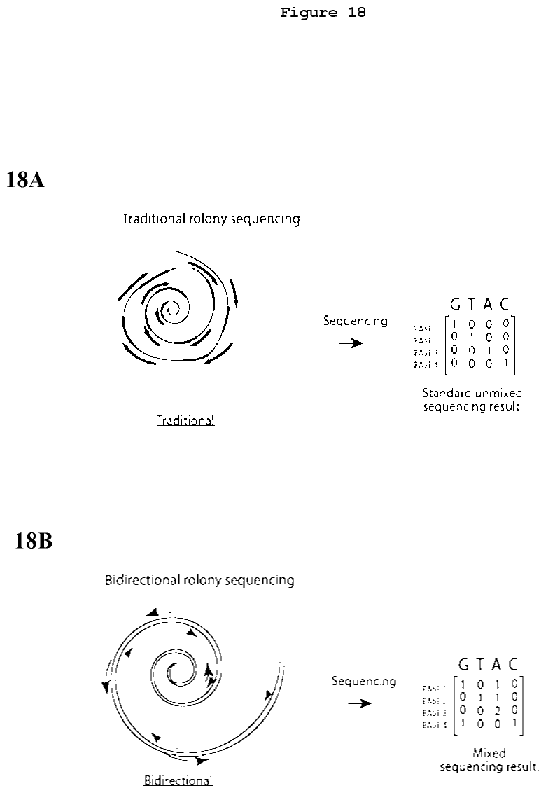

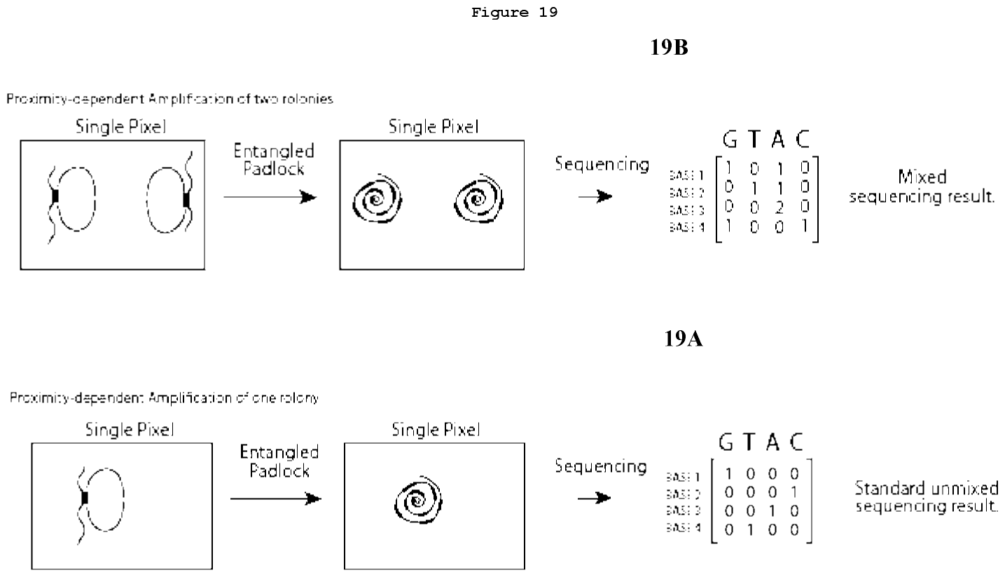

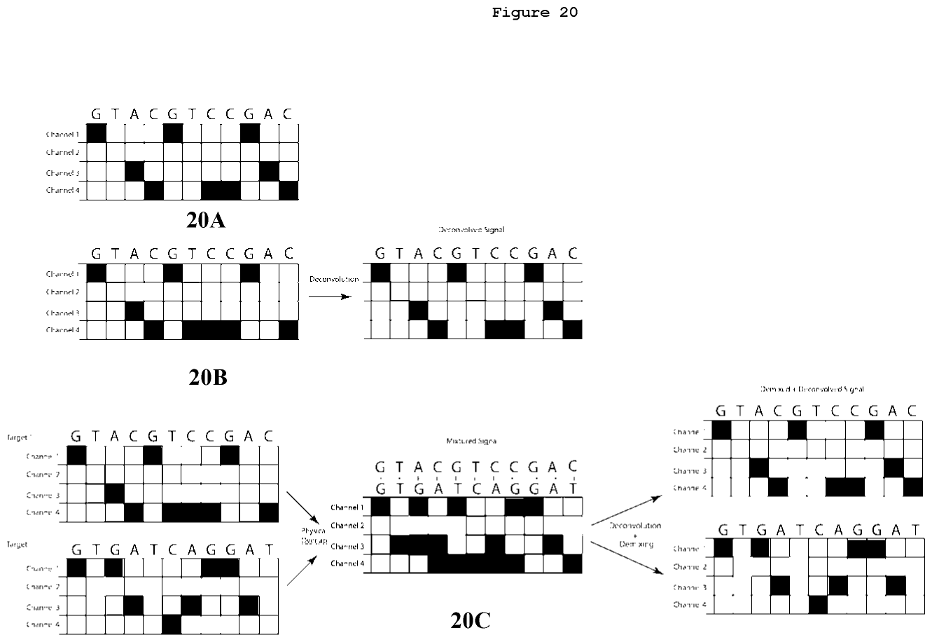

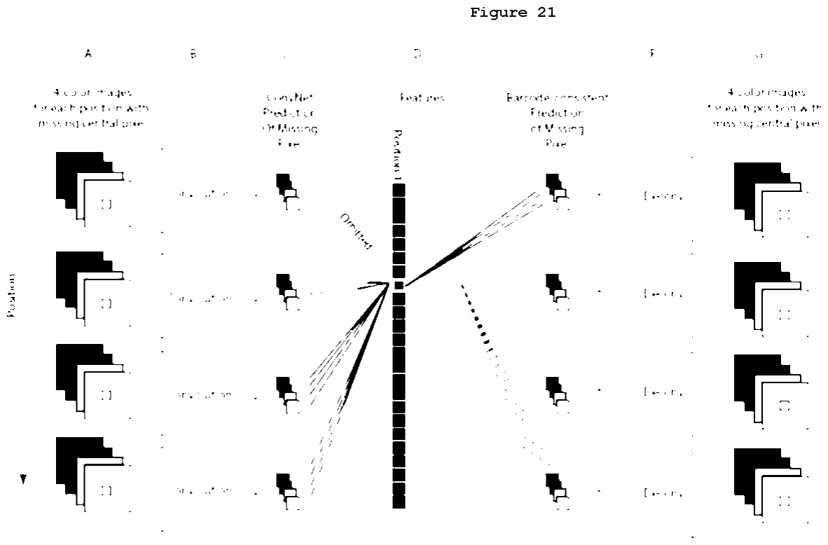

### REFERENCES

Amir and Zuk, Bacterial Community Reconstruction Using Compressed Sensing, Journal of Computational Biology VOL. 18, NO. 11.
Blanchard (2013) “Toward deterministic compressed sensing”, PNAS 110(4):1146-47.
Buschmann and Bystrykh (2013) “Levenshtein error-correcting barcodes for multiplexed DNA sequencing”, BMC Bioinformatics 14:272.
Chen et al., Nanoscale Imaging of RNA with Expansion Microscopy, Nat Methods. 2016 August; 13(8): 679-684.
Donoho and Elad (2003) “Optimally sparse representation in general (nonorthogonal) dictionaries via l¹ minimalization”, PNAS 100(5):2197-2202.
Donoho et al. (2006) “Sparse Solution of Underdetermined Linear Equations by Stagewise Orthogonal Matching Pursuit”, IEEE Transactions on Information Theory 58(2):1094-1121.
Obozinski et al. (2006) “Multi-task feature selection”, Technical Report, Department of Statistics, UC Berkley.
Olshen et al. (2004) “Circular binary segmentation for the analysis of array-based DNA copy number data”, Biostatistics 5(4):557-72.
Recht et al. (2007) “Guaranteed Minimum-Rank Solutions of Linear Matrix Equations via Nuclear Norm Minimization”, SIAM Review 52(3).
Tibshirani et al. (2005) “Sparsity and smoothness via the fused LASSO”, J. R. Statist. Soc. B 67(1):91-108.
Wang et al. (2016) “A Novel in Situ RNA Analysis Platform for Formalin-Fixed Paraffin-Embedded Tissues”, Journal of Molecular Diagnostics 14(1):22-29.

1. A method for:
I) identifying the sequence of individual target sequences which are components of a plurality of mixed sequencing signals, wherein each mixed sequencing signal represents sequencing information from the superimposition of a plurality of distinct sequencing signals, wherein the plurality of distinct sequencing signals are generated from sequencing multiple oligonucleotides in parallel, the method comprising the steps:
a) comparing each mixed sequencing signal to a sequence dictionary of reference sequences, with each mixed sequencing signal and each reference sequence represented as a sequence vector or sequence matrix; and
b) for each mixed sequencing signal, based on the representative sequence vectors or sequence vectors, identifying the set of reference sequences, which is most representative of components of the mixed sequencing signal; and
c) using: (1) relational information about the relationship between the mixed sequencing signals within the plurality of mixed sequencing signals; (2) relational information about the relationship between identified reference sequences; or (3) relational information about both the relationship between the mixed sequencing signals within the plurality of mixed sequencing signals and the relationship between identified reference sequences, thereby identifying the sequence of each individual target sequence within the plurality of mixed sequencing signals; or
II) processing sequence images to identify individual sequence vectors from a plurality of mixed sequence vectors, wherein each mixed sequence vector represents sequence information from a pixel or plurality of pixels from a series of sequence images, the method comprising the steps:
a) comparing each mixed sequence vector to a sequence vector associated with reference sequences in a sequence dictionary; and
b) identifying reference sequence vectors that are most representative of components of the mixed sequence vector; and
c) using: (1) relational information about the relationship between the mixed sequencing signals within the plurality of mixed sequencing signals; (2) relational information about the relationship between identified reference sequences; or (3) relational information about both the relationship between the mixed sequencing signals within relationship between the plurality of mixed sequencing signals and the relationship between identified reference sequences, thereby processing the sequence image vector or mixed sequence matrix into its component individual sequence vectors or sequence vectors; or
III) in-situ sequencing in a cell, comprising the steps of:
a) performing in-situ sequencing on the cell;
b) identifying images encoding mixed sequence information comprising a plurality of mixed sequencing signals, wherein the images originate from overlapping rolonies encoding different target sequences;
c) generating a plurality of mixed sequence vectors from the mixed sequencing information;
d) comparing the plurality of mixed sequence vectors to a sequence dictionary of reference sequence vectors of the cell; and
e) identifying reference sequence vectors within the sequence dictionary, which individual sequence vectors are most representative of components of the mixed sequence vector;
f) using: (1) relational information about the relationship between the mixed sequencing signals within the plurality of mixed sequencing signals; (2) relational information about the relationship between identified reference sequences; or (3) relational information about both the relationship between the mixed sequencing signals within relationship between the plurality of mixed sequencing signals and the relationship between identified reference sequences, thereby sequencing in the cell.

2. A computer-implemented process for demixing a plurality of mixed sequencing signals into their component distinct sequencing signals, the process comprising the steps:
a) obtaining a plurality of mixed sequencing signals from a process of oligonucleotide sequencing in which a plurality of distinct oligonucleotide molecules is physically or optically superimposed;
b) generating a mixed sequence vector or mixed sequence matrix from each mixed sequencing signal within the plurality of mixed sequencing signals;
c) comparing the mixed sequence vectors or mixed sequence vectors to reference sequence vectors or sequence vectors generated from a sequence dictionary of reference sequences; and
d) identifying reference sequence vectors or sequence vectors that are most representative of components of each mixed sequence vector or mixed sequence matrix
e) using: (1) relational information about the relationship between the mixed sequencing signals within the plurality of mixed sequencing signals; (2) relational information about the relationship between identified reference sequences; or (3) relational information about both the relationship between the mixed sequencing signals within relationship between the plurality of mixed sequencing signals and the relationship between identified reference sequences, thereby demixing each mixed sequence vector or mixed sequence matrix into its component individual sequence vectors or sequence vectors.

3. (canceled)

4. A non-transitory computer readable medium storing a computer program code that, when executed by one or more computers, causes the one or more computers to perform operations for processing images encoding sequencing information of a mixed sequence signal, the operations comprising:
a) detection of images comprising a plurality of mixed sequencing signals;
b) generation of a plurality of mixed sequence vectors based on the series of multiple sequencing signals;
c) comparison of each mixed sequence vector to sequence vectors drawn from a dictionary of reference sequence vectors; and
d) identification of reference sequence vectors or sequence vectors within the sequence dictionary, which reference sequence vectors or sequence vectors are most representative of components of the mixed sequence vector, thereby processing the images encoding sequencing information of a mixed sequence vector into individual sequence vectors; and
e) using: (1) relational information about the relationship between the mixed sequencing signals within the plurality of mixed sequencing signals; (2) relational information about the relationship between identified reference sequences; or (3) relational information about both the relationship between the mixed sequencing signals within relationship between the plurality of mixed sequencing signals and the relationship between identified reference sequences, thereby processing the images encoding sequencing information of a mixed sequence signal.

5. (canceled)

6. The method of claim 1, wherein the sequencing information is generated from a next-generation sequencing, or a high-throughput sequencing platform.

7. The method of claim 1, wherein the sequencing information is generated by:
a) a sequencing by synthesis (SBS) method;
b) a sequencing by ligation method; or
c) fluorescent in-situ sequencing (FISSEQ).

8-9. (canceled)

10. The method of claim 1, wherein the identified individual sequence vectors contributing to the mixed sequence vector form a sparse solution.

11. The method of claim 1, wherein the sparsest solution is identified by determining the solution with the smallest L1 norm of
argmin(x)|x|_1 s.t. s=Ax,
in the absence of noise, or
argmin(x)|x|_1 s.t.|Ax−s|<eps,
accounting for noise, where s=weighted sum of the elements, or columns, of A, A=sequence dictionary, x=set of weights or loading.

12. The method of claim 1, wherein the sequencing information of the mixed sequence vector is generated from:
a) optically overlapping sequencing signals originating from multiple distinct oligonucleotides; or
b) simultaneously sequencing multiple regions of an oligonucleotide.

13. (canceled)

14. The method of claim 1, wherein the sequencing signal across neighboring pixels is assumed to be similar (spatial smoothness) and use the same sequence dictionary elements, enabling multi-pixel decoding by determining the solution of
min(|X|) s.t.∥AX−S∥<epsilon AND f(X)<epsilon,
where S=a measurement matrix that is the repeated measurement vector s, X=a weight matrix of the non-zero weights shared across pixels of weight vector x, A=sequence dictionary, f(X)=a measure of the spatial variation in X.

15. The method of claim 1, wherein the comparison between a mixed sequence vector to a reference sequence dictionary is an application of:
a) a compressed sensing algorithm or neural network; or
b) an algorithm selected from the group consisting of: regression, constrained regression, least absolute shrinkage and selection operator (LASSO), combinatorial theory, convex optimization, approximate message passing, belief propagation or a convex or nonconvex solver of any kind.

16. (canceled)

17. The method of claim 1, wherein the reference sequence dictionary is not known in advance, or is only partially known in advance, and wherein the recovery of the reference sequence dictionary as well as potential target sequences contained in each of a plurality of mixed sequencing signals is an application of an algorithm selected from the group consisting of: non-negative matrix factorization, independent components analysis, matrix factorization, Bayesian learning, neural network, tensor decomposition, or deep learning.

18. The method of claim 1, wherein the average distance between overlapping rolonies are within at least 0.5.m 1.m, 2.m of its nearest neighboring rolony.

19. The method of claim 1, wherein rolonies are considered to overlap if, for at least 5%, 10%, 25%, 50%, 90%, or 100% of rolonies, at least 5%, 10%, 25%, 50%, 90%, or 100% of the pixels imaged for a rolony overlap with pixels from another rolony.

20. The method of claim 1, wherein the identified reference sequence vectors are sufficient to explain the variability of the mixed sequence vector with an error less than 90%, 75%, 50%, 25%, 10%, 5% or less.

21. The method of claim 1, wherein the sparsity of the solution is quantified by the measurement of the norm of the recovered loading vector.

22. The method of claim 1, wherein the sequence dictionary contains sequence vectors representing genomic, transcriptomic, metagenomic, or barcode sequences.

23. The method of claim 1, wherein the sequence dictionary is not fully known in advance, but is recovered by joint analysis of a plurality of multiple sequence signals.

24. The method of claim 1, wherein the sequence dictionary A and the weight matrix X can be learned by non-negative matrix factorization of the mixed fluorescence signal between neighboring pixels, such that
S=WH,
where the signal S can be reconstructed by identifying the set of sparse components W that can be combined via the weight matrix H, and all entries in W and H are constrained to be non-negative.
